(19) United States
(12) Patent Application Publication
(10) Pub. No.: US 2012/0302756 A1
(43) Pub. Date: Nov. 29, 2012

(54) PROCESS FOR SYNTHESIS OF 2-SUBSTITUTED PYRROLIDINES AND PIPERADINES

(76) Inventors: Rajender Reddy Leleti, East Hanover, NJ (US); Yugang Liu, East Hanover, NJ (US); Mahavir Prashad, East Hanover, NJ (US)

(21) Appl. No.: 13/576,427

(22) PCT Filed: Feb. 17, 2011
(86) PCT No.: PCT/US11/25191

§ 371 Date: Aug. 1, 2012

Related U.S. Application Data
(60) Provisional application No. 61/306,069, filed on Feb. 19, 2010.

(51) Int. Cl. C07D 211/96; C07D 203/24

(52) U.S. Cl. 546/184; 548/965

(57) ABSTRACT

The present invention provides a highly efficient, versatile one-step process for asymmetric synthesis of either diastereomer of 2-substituted pyrrolidines from a single starting material with excellent yields and high diastereoselectivety. Also provided is a method for the asymmetric synthesis of both diastereomers of 2-substituted piperidines with good yields and excellent diastereoselectivety. Diasteroselectivity is controlled effectively by choice of reducing agent.

### BACKGROUND OF THE INVENTION

Diastereomerically pure 2-substituted pyrrolidines and 2-substituted piperadines are ubiquitous structural motifs found in a wide variety of natural products and pharmaceutical drugs. Over 4,000 compounds containing 2-substituted pyrrolidines and over 200 compounds containing 2-substituted piperadines are currently in advanced stage of biological testing. (See CMDL Drug Data Report, M.I.S. Inc., San Leandro, Calif. (2009).) In addition, these compounds are effective chiral controllers and thus play an important role in asymmetric synthesis. As a result, the synthesis of these compounds has been an active area of research.

Known synthetic methods of these privileged structures suffer from either long synthetic sequences, low yields, lack of generality or modest selectivity. The classical approach to obtain diastereomerically pure 2-substituted pyrrolidines and 2-substituted piperadines is by resolution of the racemate via diastereoselective salt formation. However, the maximum theoretical yield for resolution is only 50%. Campos et al., Journal of the American Chemical Society 128, 3538-3539 (2006), have reported an elegant method for asymmetric synthesis of 2-substituted pyrrolidines in a highly enantioselective manner. This method employs (−)-sparteine mediated enantioselective lithiation of N-Boc-pyrrolidine, followed by transmetalation and palladium (Pd)-catalyzed Negishi coupling. This method is limited to the synthesis of (R)-aryl pyrrolidines due to the lack of inexpensive alternatives for (+)-sparteine. In addition, it utilizes sec-butyllithium (s-BuLi), which is a pyrophoric liquid and restricted from use on a large scale.

Recently, Reddy et al, Chemical Communications, 46(2), 222-224 (2010), reported an asymmetric synthesis of 2-substituted pyrrolidines by addition of Grignard reagents to γ-chloro N-tert-butanesulfinyl aldimine followed by base catalyzed cyclization. This method provides either diastereomer of 2-substituted pyrrolidines by changing the chiral starting material. While the diastereoselective reduction of N-tert-butanesulfinyl ketimines is well established, the asymmetric reductive cyclization of γ-chloro N-tert-butanesulfinyl ketimines to selectively produce either diastereomer of 2-substituted pyrrolidines has not been achieved heretobefore. The development of an asymmetric synthesis to provide either diastereomer of 2-substituted pyrrolidines and 2-substituted piperadines from the same starting material would therefore be a great improvement over the art.

The present invention provides a versatile and practical method for diastereoselective reductive cyclization of (S_{S})-γ-chlorinated N-tert-butanesulfinyl ketimines to give either diastereomer of 2-substituted pyrrolidines in a single step with high yields. This method may further be used for diastereoselective reductive cyclization of (S_{R})-γ-chlorinated N-tert-butanesulfinyl ketimines to give either diastereomer of 2-substituted pyrrolidines in a single step with high diastereoselectivity.

The present invention also provides a versatile and practical method for diastereoselective reductive cyclization of (S_{S})-δ-chlorinated N-tert-butanesulfinyl ketimines to give either diastereomer of 2-substituted piperidines in a single step with high yields. This method may further be used for diastereoselective reductive cyclization of (S_{R})-δ-chlorinated N-tert-butanesulfinyl ketimines to give either diastereomer of 2-substituted piperidines in a single step with high diastereoselectivity.

### SUMMARY OF THE INVENTION

The present invention provides a one step process for producing either diastereomer of 2-substituted pyrrolidines from the same starting material. Reductive cyclization of (S_{S}-γ-chloro-N-tert-butanesulfinyl ketimines with a reducing agent selected from the group consisting of lithiumtriethylborohydride (LiBHEt₃) and L-Selectride in a suitable solvent at a temperature of about −78 to 23 C.° affords (S_{S},R)—N-tert-butanesulfinyl-2-substituted pyrrolidines in excellent yields (88-98%) and with high diastereoselectivity (99:1). Reductive cyclization of (S_{S})-γ-chloro-N-tert-butanesulfinyl ketimines in a suitable solvent with diisobutylaluminum-hydride (DIBAL-H) in the presence of a strong base such as lithium hexamethyldisilazide and at the temperature range of about −78° C. to about 0° C. affords (S_{S},S)-2-substituted pyrrolidines in good yields (87-98%) and with high diastereoselectivity (1:99).

In an embodiment of the present invention, reductive cyclization of (S_{R})-γ-chloro-N-tert-butanesulfinyl ketimines with a reducing agent selected from the group consisting of lithiumtriethylborohydride (LiBHEt₃) and L-Selectride in a suitable solvent at a temperature of about −78 to 23 C.° may be used to produce (S_{R},S)—N-tert-butanesulfinyl-2-substituted pyrrolidines with high diastereoselectivity. Reductive cyclization of (S_{R})-γ-chloro-N-tert-butanesulfinyl ketimines in a suitable solvent with diisobutylaluminum-hydride (DIBAL-H) in the presence of a strong base such as lithium hexamethyldisilazide (LiHMDS) and at the temperature range of about −78° C. to about 0° C. affords (S_{R},R)-2-substituted pyrrolidines with high diastereoselectivity.

The present invention further provides a one step process for producing either diastereomer of 2-substituted piperidines from the same starting material. Reductive cyclization of (S_{S})-δ-chloro-N-tert-butanesulfinyl ketimine with lithiumtriethylborohydride (LiBHEt₃) in a suitable solvent at −78 to about 23° C. affords the (S_{S},R)—N-tert-butanesulfinyl-2-substituted piperidines in excellent yield (98%) and with high diastereoselectivity (99:1). Reductive cyclization of (S_{S})-δ-chloro-N-tert-butanesulfinyl ketimine in a suitable solvent with diisobutylaluminum-hydride (DIBAL-H) in the presence of a strong base such as lithium hexamethyldisilazide (LiHMDS) at about −78° C. to about 0° C. affords the (S_{S},S)—N-tert-butanesulfinyl-2-substituted piperidines in good yield (98%) and with high diastereoselectivity (1:99).

In an embodiment of the present invention, reductive cyclization of (S_{R})-δ-chloro-N-tert-butanesulfinyl ketimine with lithiumtriethylborohydride (LiBHEt₃) in a suitable solvent at −78 to about 23° C. may be used to produce the (S_{R},S)—N-tert-butanesulfinyl-2-substituted piperidines with high diastereoselectivity. Reductive cyclization of (S_{R})-δ-chloro-N-tert-butanesulfinyl ketimine in a suitable solvent with diisobutylaluminum-hydride (DIBAL-H) in the presence of a strong base such as lithium hexamethyldisilazide (LiHMDS) at about −78° C. to about 0° C. may be used to produce the (S_{R},R)—N-tert-butanesulfinyl-2-substituted piperidines with high diastereoselectivity.

In an alternate embodiment, the 2-position on either the pyrrolidine or piperidine ring is substituted with various aromatic, heteromatic or aliphatic substituents. Preferably, the 2-position on either the pyrrolidine or piperidine ring is substituted with a substituent selected from the group consisting of 4-BrC₆H₄, C₆H₅, 4-MeC₆H₄, 4-MeOC₆H₄, 4-tBuC₆H₄, 4-HOC₆H₄, 3-MeOC₆H₄, 4-ClC₆H₄, 4-FC₆H₄, 2-thienyl, C₆H₁₁ and Me.

In a further embodiment, the N-tert-butanesulfinyl-2-substituted pyrrolidines and piperidines may be deprotected using a mild acid to cleave the sulfinyl group, yielding the corresponding enantiomers of 2-substituted pyrrolidines or diastereomers of 2-substituted piperidines in quantative yield.

### BRIEF DESCRIPTION OF THE DRAWINGS

FIG. 1 is a drawing reflecting the X-ray crystal structure of (R)-1-((S)-tert-butylsulfinyl)-2-(4-bromophenyl)pyrrolidine (2a).

### DETAILED DESCRIPTION OF THE INVENTION

The present invention provides a versatile one-step process for asymmetric synthesis of either diastereomer of 2-substituted pyrrolidines from the same starting material with excellent yields and high diastereoselectivety. The method may also be applied for the asymmetric synthesis of either diastereomer of 2-substituted piperidines with good yields and excellent diastereoselectivity.

In accordance with the present invention, there is provided a method for preparing a diastereomerically pure (S_{S},R)—N-tert-butanesulfinyl-2-substituted pyrrolidines comprising reductive cyclization of (S_{S})-γ-chloro-N-tert-butanesulfinyl ketimines in a suitable solvent (e.g., tetrahyrofuran or toluene) with a reducing agent selected from the group consisting of lithiumtriethylborohydride (LiBHEt₃) and L-Selectride at a temperature of about −78 to 23° C. This method affords (S_{S},R)—N-tert-butanesulfinyl-2-substituted pyrrolidines in excellent yields (88-98%) and with high diastereoselectivity (99:1). The diastereoselectivity is controlled effectively by the choice of reducing agent. Thus, the corresponding epimers of (S_{S},S)-2-substituted pyrrolidines may be synthesized in good yields (87-98%) and with high diastereoselectivity (1:99) by simply switching the reducing agent from LiBHEt₃ or L-Selectride to diisobutylaluminum-hydride (DIBAL-H) in the presence of a strong base such as lithium hexamethyldisilazide (LiHMDS). Deprotection of N-tert-butanesulfinyl-2-substituted pyrrolidines using a mild acid to cleave the sulfinyl group gives the corresponding enantiomers of 2-substituted pyrrolidines in quantative yield.

Thus, the present invention provides a versatile and practical method for diastereoselective reductive cyclization of (S_{S})-γ-chlorinated N-tert-butanesulfinyl ketimines (3) to give either diastereomer of 2-substituted pyrrolidines (4 or 5) in a single step with high yields (Scheme 1). The tert-butanesulfinyl group not only induces excellent diastereoselectivity but also serves as an efficient low molecular weight protecting group for the nitrogen for future modifications of the 2-substituted pyrrolidines, if needed.

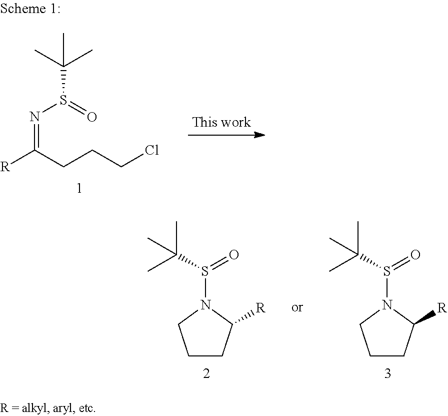

In one embodiment of the present invention, there is provided a method for preparing a diastereomerically pure (S_{S},R)-2-substituted pyrrolidine of Formula (Ia)

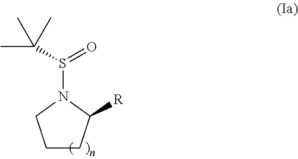

comprising reductive cyclization of an (S_{S})-γ-chloro-N-tert-butanesulfinyl ketimine of Formula (II)

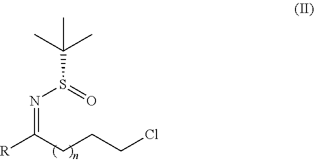

in a suitable solvent with a reducing agent selected from the group consisting of lithiumtriethylborohydride (LiBHEt₃) and L-Selectride, at a temperature of about −78 to 23° C. for a period of about 3 hours to 12 hours, followed by warming to room temperature and stirring for a sufficient period of time. In both Formula (Ia) and Formula (II), n is 0 or 1, and R is the same and one of: alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide or sulfoxide.

Preferably R is selected from the group consisting of: 4-BrC₆H₄, C₆H₅, 4-MeC₆H₄, 4-MeOC₆H₄, 4-tBuC₆H₄, 4-HOC₆H₄, 3-MeOC₆H₄, 4-ClC₆H₄, 4-FC₆H₄, 2-thienyl, C₆H₁₁ and Methyl (Me).

The R group of Formula (Ia) and Formula (II) may be further substituted with a substituent selected from the group consisting of substituted alkyl, unsubstituted alkyl, substituted alkenyl, unsubstituted alkenyl, substituted alkenyl, unsubstituted alkenyl, alkynyl, alkenylhalogens, hydroxyls, alkoxy, alkenoxy, cycloalkoxy, aryloxy, aralkyloxy, heterocyclyloxy, heterocyclylalkoxy, carbonyls (oxo), carboxyls, urethanes, oximes, hydroxylamines, alkoxyamines, aralkoxyamines, thiols, sulfides, sulfoxides, sulfonamide, amines, N-oxides, hydrazines, hydrazides, hydrazones, azides, amides, ureas, amidines, guanidines, enamines, imides, and nitro groups.

The method of the present invention employs the reducing agent LiBHEt₃ or L-Selectride in an amount that is at least 1 molar equivalent of the (S_{S})-γ-chloro-N-tert-butanesulfinyl ketimine. It is understood that the reducing agent LiBHEt₃ or L-Selectride may be present in an amount greater than 1 molar equivalent of the (S_{S})-γ-chloro-N-tert-butanesulfinyl ketimine.

Examples of suitable solvents that may be used with the reducing agents lithiumtriethylborohydride (LiBHEt₃) or L-Selectride in accordance with the present invention include dichloromethane, dichloroethane, chloroform, carbon tetrachloride, tetrahydrofuran (THF), methyl tertbutyl ether, diisopropyl ether, diethyl ether, toluene, chlorobenzene, acetonitrile and the like or mixtures thereof. Preferred is tetrahydrofuran (THF) and toluene, and most preferred is THF.

A sufficient period of time for stirring at room temperature (20-25° C.) is at least about 1 hour.

If desired, the sulfinyl group may be cleaved from the diastereomerically pure (S_{S}R)-2-substituted pyrrolidine of Formula (Ia) to yield the corresponding 2-substituted pyrrolidine enantiomer. A mild acid may be used to achieve removal of the sulfinyl group. Examples of mild acids include, but are not limited to, HCl, trifluoroacetic acid (TFA) and sulfuric acid. Preferably, HCl is used. Most preferably, 4N HCl in dioxane and MeOH is used for deprotection.

In a further embodiment, there is provided a method for preparing a diastereomerically pure (S_{S},R)-2-substituted pyrrolidine of Formula (Ia) as set forth above comprising reductive cyclization of an (S_{S})-γ-chloro-N-tert-butanesulfinyl ketimine of Formula II as set forth above in the solvent THF with the reducing agent lithiumtriethylborohydride (LiBHEt₃) or L-Selectride, at a temperature of about −78° C. for a period of about 3 hours, followed by warming to room temperature and stirring for a sufficient period of time. In both Formula (Ia) and Formula (II), n is 0 or 1, and R is as defined above.

The present invention also provides a method for preparing a diastereomerically pure (S_{S}S)-2-substituted pyrrolidine of Formula (1b):

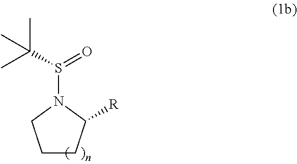

comprising reductive cyclization of (S_{S})-γ-chloro-N-tert-butanesulfinyl ketimines of Formula (II)

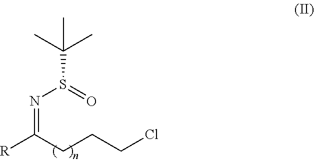

in a suitable solvent with the reducing agent diisobutylaluminum-hydride (DIBAL-H) in the presence of a strong base selected from the group consisting of lithium hexamethyldisilazide (LiHMDS), butyllithium (BuLi), sodium hydride (NaH), triethylamine (CH₂CH₃)₃N or NEt₃) and potassium hydroxide (KOH), wherein the suitable solvent is selected from the group consisting of:
(i) toluene or tetrahydrofuran when the strong base is LiHMDS,
(ii) THF when the strong base is BuLi or NaH, and
(iii) acetonitrile when the strong base is NEt₃, and
(iv) THF—H₂O when the strong base is KOH,
   and at a temperature of about ⁻78 to 0° C. for a period of about 3 hours to 12 hours, followed by warming to room temperature and stirring for a sufficient period of time. In both Formula (Ib) and Formula (II), n is 0 or 1, and R is the same and one of: alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, or sulfoxide.

Preferably R is an aromatic, heteroaromatic, and aliphatic substituent selected from the group consisting of: 4-BrC₆H₄, C₆H₅, 4-MeC₆H₄, 4-MeOC₆H₄, 4-tBuC₆H₄, 4-HOC₆H₄, 3-MeOC₆H₄, 4-ClC₆H₄, 4-FC₆H₄, 2-thienyl, C₆H₁₁ and Methyl (Me).

The R group of Formula (Ib) and (II) may be further substituted with a substituent selected from the group consisting of substituted alkyl, unsubstituted alkyl, substituted alkenyl, unsubstituted alkenyl, substituted alkenyl, unsubstituted alkenyl, alkynyl, alkenylhalogens, hydroxyls, alkoxy, alkenoxy, cycloalkoxy, aryloxy, aralkyloxy, heterocyclyloxy, heterocyclylalkoxy, carbonyls (oxo), carboxyls, urethanes, oximes; hydroxylamines, alkoxyamines, aralkoxyamines, thiols, sulfides, sulfoxides, sulfonamides, amines, N-oxides, hydrazines, hydrazides, hydrazones, azides, amides, ureas, amidines, guanidines, enamines, imides, and nitro groups.

The strong base used in the method of the present invention is selected from the group consisting of lithium hexamethyldisilazide (LiHMDS), butyllithium (BuLi), sodium hydride (NaH), triethylamine (CH₂CH₃)₃N or NEt₃) and potassium hydroxide (KOH). The preferred strong base is lithium hexamethyldisilazide (LiHMDS).

The method of the present invention employs the reducing agent DIBAL-H and the strong base as defined herein in an amount that is at least 1 molar equivalent of the DIBAL-H and at least one molar equivalent of the strong base as defined herein as compared to the (S_{S})-γ-chloro-N-tert-butanesulfinyl ketimine. It is understood that the reducing agent DIBAL-H and the strong base as defined herein may be present in an amount greater than 1 molar equivalent of the (S_{S})-γ-chloro-N-tert-butanesulfinyl ketimine.

The suitable solvent used in the method of the present invention is selected from the group consisting of:
(v) toluene or tetrahydrofuran when the strong base is LiHMDS,
(vi) THF when the strong base is BuLi or NaH, and
(vii) acetonitrile when the strong base is NEt₃, and
(viii) THF—H₂O when the strong base is KOH.
   The preferred solvent is toluene when the strong base is LiHMDS.

A sufficient period of time for stirring at room temperature (20-25° C.) is at least about 2 hours. However, longer periods of time may be used, e.g., from about 2 hours to upward of 12 hours.

If desired, the sulfinyl group may be cleaved from the diastereomerically pure (S_{S}S)-2-substituted pyrrolidine of Formula (Ib) as set forth above to yield the corresponding 2-substituted pyrrolidine enantiomer. A mild acid may be used to achieve removal of the sulfinyl group. Examples of mild acids include, but are not limited to, HCl, trifluoroacetic acid, and sulfuric acid Preferably, HCl is used. Most preferably, 4N HCl in dioxane and MeOH is used for deprotection.

In a further embodiment, there is provided a method for preparing a diastereomerically pure (S_{S}S)-2-substituted pyrrolidine of Formula (Ib) as set forth above comprising reductive cyclization of (S_{S})-γ-chloro-N-tert-butanesulfinyl ketimines of Formula (II) as set forth above in the suitable solvent toluene with the reducing agent diisobutylaluminum-hydride (DIBAL-H) in the presence of a strong base LiHMDS and at a temperature of about ⁻78° C. for a period of about 3 hours, followed by warming to room temperature and stirring for a sufficient period of time.

It is further understood that the present invention provides a method for preparing a diastereomerically pure (S_{R},S)—N-tert-butanesulfinyl-2-substituted pyrrolidines and (S_{R},R)—N-tert-butanesulfinyl-2-substituted pyrrolidines comprising reductive cyclization of (S_{R})-γ-chloro-N-tert-butanesulfinyl ketimines by applying the same process as set forth above.

In one embodiment of the present invention, there is provided a method for preparing a diastereomerically pure (S_{R},S)-2-substituted pyrrolidine of Formula (Ic)

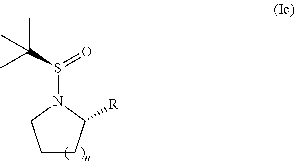

comprising reductive cyclization of an (S_{R})-γ-chloro-N-tert-butanesulfinyl ketimine of Formula (IIa)

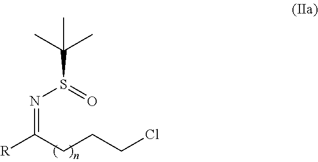

in a suitable solvent with a reducing agent selected from the group consisting of lithiumtriethylborohydride (LiBHEt₃) or L-Selectride, at a temperature of about −78 to 23° C. for a period of about 3 hours to 12 hours, followed by warming to room temperature and stirring for a sufficient period of time. In both Formula (Ic) and Formula (IIa), n is 0 or 1, and R is the same and one of: alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide or sulfoxide.

Preferably R is an aromatic, heteroaromatic, and aliphatic substituent selected from the group consisting of: 4-BrC₆H₄, C₆H₅, 4-MeC₆H₄, 4-MeOC₆H₄, 4-tBuC₆H₄, 4-HOC₆H₄, 3-MeOC₆H₄, 4-ClC₆H₄, 4-FC₆H₄, 2-thienyl, C₆H₁₁ and Methyl (Me).

The R group of Formula (Ic) and Formula (IIa) may be further substituted with a substituent selected from the group consisting of substituted alkyl, unsubstituted alkyl, substituted alkenyl, unsubstituted alkenyl, substituted alkenyl, unsubstituted alkenyl, alkynyl, alkenylhalogens, hydroxyls, alkoxy, alkenoxy, cycloalkoxy, aryloxy, aralkyloxy, heterocyclyloxy, heterocyclylalkoxy, carbonyls (oxo), carboxyls, urethanes, oximes, hydroxylamines, alkoxyamines, aralkoxyamines, thiols, sulfides, sulfoxides, sulfonamide, amines, N-oxides, hydrazines, hydrazides, hydrazones, azides, amides, ureas, amidines, guanidines, enamines, imides, and nitro groups.

The method of the present invention employs the reducing agent LiBHEt₃ or L-Selectride in an amount that is at least 1 molar equivalent of the (S_{R})-γ-chloro-N-tert-butanesulfinyl ketimine. It is understood that the reducing agent LiBHEt₃ or L-Selectride may be present in an amount greater than 1 molar equivalent of the (S_{R})-γ-chloro-N-tert-butanesulfinyl ketimine.

Examples of suitable solvents that may be used with the reducing agents lithiumtriethylborohydride (LiBHEt₃) or L-Selectride in accordance with the present invention include dichloromethane, dichloroethane, chloroform, carbon tetrachloride, tetrahydrofuran (THF), methyl tertbutyl ether, diisopropyl ether, diethyl ether, toluene, chlorobenzene, acetonitrile and the like or mixtures thereof. Preferred is tetrahydrofuran (THF) and toluene, and most preferred is THF.

A sufficient period of time for stirring at room temperature (20-25° C.) is at least about 1 hour.

If desired, the sulfinyl group may be cleaved from the diastereomerically pure (S_{S},R)-2-substituted pyrrolidine of Formula (Ic) to yield the corresponding 2-substituted pyrrolidine enantiomer. A mild acid may be used to achieve removal of the sulfinyl group. Examples of mild acids include, but are not limited to, HCl, trifluoroacetic acid (TFA) and sulfuric acid. Preferably, HCl is used. Most preferably, 4N HCl in dioxane and MeOH is used for deprotection.

In a further embodiment, there is provided a method for preparing a diastereomerically pure (S_{R}S)-2-substituted pyrrolidine of Formula (Ic) as set forth above comprising reductive cyclization of an (S_{R})-γ-chloro-N-tert-butanesulfinyl ketimine of Formula II as set forth above in the solvent THF with the reducing agent lithiumtriethylborohydride (LiBHEt₃) or L-Selectride, at a temperature of about −78° C. for a period of about 3 hours, followed by warming to room temperature and stirring for a sufficient period of time. In both Formula (Ic) and (IIa), n is 0 or 1, and R is as defined above.

The present invention also provides a method for preparing a diastereomerically pure (S_{R},R)-2-substituted pyrrolidine of Formula (Id):

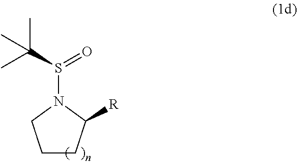

comprising reductive cyclization of (S_{R})-γ-chloro-N-tert-butanesulfinyl ketimines of Formula (IIa)

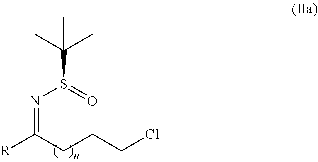

in a suitable solvent with the reducing agent diisobutylaluminum-hydride (DIBAL-H) in the presence of a strong base selected from the group consisting of lithium hexamethyldisilazide (LiHMDS), butyllithium (BuLi), sodium hydride (NaH), triethylamine (CH₂CH₃)₃N or NEt₃) and potassium hydroxide (KOH), wherein the suitable solvent is selected from the group consisting of:
(i) toluene or tetrahydrofuran when the strong base is LiHMDS,
(ii) THF when the strong base is BuLi or NaH, and
(iii) acetonitrile when the strong base is NEt₃, and
(iv) THF—H₂O when the strong base is KOH,
   and at a temperature of about ⁻78 to 0° C. for a period of about 3 hours to 12 hours, followed by warming to room temperature and stirring for a sufficient period of time. In both Formula (Id) and Formula (IIa), n is 0 or 1, and R is the same and one of: alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, or sulfoxide.

Preferably R is selected from the group consisting of: 4-BrC₆H₄, C₆H₅, 4-MeC₆H₄, 4-MeOC₆H₄, 4-tBuC₆H₄, 4-HOC₆H₄, 3-MeOC₆H₄, 4-ClC₆H₄, 4-FC₆H₄, 2-thienyl, C₆H₁₁ and Me.

The R group of Formula (Id) and (IIa) may be further substituted with a substituent selected from the group consisting of substituted alkyl, unsubstituted alkyl, substituted alkenyl, unsubstituted alkenyl, substituted alkenyl, unsubstituted alkenyl, alkynyl, alkenylhalogens, hydroxyls, alkoxy, alkenoxy, cycloalkoxy, aryloxy, aralkyloxy, heterocyclyloxy, heterocyclylalkoxy, carbonyls (oxo), carboxyls, urethanes, oximes; hydroxylamines, alkoxyamines, aralkoxyamines, thiols, sulfides, sulfoxides, sulfonamides, amines, N-oxides, hydrazines, hydrazides, hydrazones, azides, amides, ureas, amidines, guanidines, enamines, imides, and nitro groups.

The strong base used in the method of the present invention is selected from the group consisting of lithium hexamethyldisilazide (LiHMDS), butyllithium (BuLi), sodium hydride (NaH), triethylamine (CH₂CH₃)₃N or NEt₃) and potassium hydroxide (KOH). The preferred strong base is lithium hexamethyldisilazide (LiHMDS).

The method of the present invention employs the reducing agent DIBAL-H and the strong base as defined herein in an amount that is at least 1 molar equivalent of the DIBAL-H and at least one molar equivalent of the strong base as defined herein as compared to the (S_{R})-γ-chloro-N-tert-butanesulfinyl ketimines. It is understood that the reducing agent DIBAL-H and the strong base as defined herein may be present in an amount greater than 1 molar equivalent of the (S_{R})-γ-chloro-N-tert-butanesulfinyl ketimines.

The suitable solvent used in the method of the present invention is selected from the group consisting of:
(i) toluene or tetrahydrofuran when the strong base is LiHMDS,
(ii) THF when the strong base is BuLi or NaH, and
(iii) acetonitrile when the strong base is NEt₃, and
(iv) THF—H₂O when the strong base is KOH.
   The preferred solvent is toluene when the strong base is LiHMDS.

A sufficient period of time for stirring at room temperature (20-25° C.) is at least about 2 hours. However, longer periods of time may be used, e.g., from about 2 hours to upward of 12 hours.

If desired, the sulfinyl group may be cleaved from the diastereomerically pure (S_{S}S)-2-substituted pyrrolidine of Formula (Id) to yield the corresponding 2-substituted pyrrolidine enantiomer. A mild acid may be used to achieve removal of the sulfinyl group. Examples of mild acids include, but are not limited to, HCl, trifluoroacetic acid, and sulfuric acid Preferably, HCl is used. Most preferably, 4N HCl in dioxane and MeOH is used for deprotection.

In a further embodiment, there is provided a method for preparing a diastereomerically pure (S_{R},R)-2-substituted pyrrolidine of Formula (Id) as set forth above comprising reductive cyclization of (S_{R})-γ-chloro-N-tert-butanesulfinyl ketimines of Formula (II) as set forth above in the suitable solvent toluene with the reducing agent diisobutylaluminum-hydride (DIBAL-H) in the presence of a strong base LiHMDS and at a temperature of about ⁻78° C. for a period of about 3 hours, followed by warming to room temperature and stirring for a sufficient period of time.

In accordance with the present invention, there is provided a method for preparing a diastereomerically pure (S_{S},R)—N-tert-butanesulfinyl-2-substituted piperidines comprising reductive cyclization of (S_{S})-δ-chloro-N-tert-butanesulfinyl ketimine in a suitable solvent with the reducing agent lithiumtriethylborohydride (LiBHEt₃) or L-Selectride at a temperature of about −78 to 23° C. This method affords (S_{S},R)—N-tert-butanesulfinyl-2-substituted piperidines in excellent yield (98%) and with high diastereoselectivity (99:1). The diastereoselectivity is similarly controlled effectively by the choice of reducing agent. Thus, the corresponding epimers of (S_{S},S)—N-tert-butanesulfinyl-2-substituted piperidines may be synthesized in good yields (98%) and with high diastereoselectivity (1:99) by switching the reducing agent from LiBHEt₃ to diisobutylaluminum-hydride (DIBAL-H) in the presence of a strong base such as lithium hexamethyldisilazide (LiHMDS) at about −78° C. to about 0° C. Deprotection of N-tert-butanesulfinyl-2-substituted piperadines using a mild acid to cleave the sulfinyl group gives the corresponding enantiomers of 2-substituted piperadines in quantative yield. This method is particularly effective for a variety of substrates including aromatic, heteroaromatic, and aliphatic substituents.

In one embodiment of the present invention, there is provided a method for preparing a diastereomerically pure (S_{S},R)-2-substituted piperidine of Formula (IIIa):

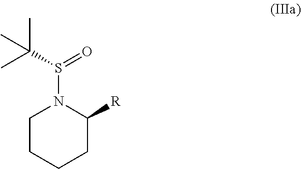

comprising reductive cyclization of (S_{S})-δ-chloro-N-tert-butanesulfinyl ketimine of Formula (IV)

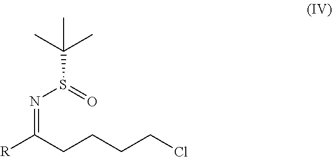

in a suitable solvent with a reducing agent selected from the group consisting of lithiumtriethylborohydride (LiBHEt₃) or L-Selectride at a temperature of about ⁻78 to 23° C. for about 3 hours to 12 hours, followed by warming to about room temperature and stirring for a sufficient period of time. In both Formula (IIIa) and (IV), R is the same and one of: alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, or sulfoxide.

Preferably R is a phenyl group.

The R group of Formula (IIIa) or (IV) may be further substituted with a substituent selected from the group consisting of substituted alkyl, unsubstituted alkyl, substituted alkenyl, unsubstituted alkenyl, substituted alkenyl, unsubstituted alkenyl, alkynyl, alkenylhalogens, hydroxyls, alkoxy, alkenoxy, cycloalkoxy, aryloxy, aralkyloxy, heterocyclyloxy, heterocyclylalkoxy, carbonyls (oxo), carboxyls, urethanes, oximes, hydroxylamines, alkoxyamines, aralkoxyamines, thiols, sulfides, sulfoxides, sulfonamides, amines, N-oxides, hydrazines, hydrazides, hydrazones, azides, amides, ureas, amidines, guanidines, enamines, imides, and nitro groups.

The method of the present invention employs the reducing agent LiBHEt₃ or L-Selectride in an amount that is at least 1 molar equivalent of the (S_{S})-δ-chloro-N-tert-butanesulfinyl ketimine. It is understood that the reducing agent LiBHEt₃ or L-Selectride may be present in an amount greater than 1 molar equivalent of the (S_{S})-δ-chloro-N-tert-butanesulfinyl ketimine.

Examples of suitable solvents that may be used with the reducing agents lithiumtriethylborohydride (LiBHEt₃) or L-Selectride in accordance with the present invention include dichloromethane, dichloroethane, chloroform, carbon tetrachloride, tetrahydrofuran (THF), methyl tertbutyl ether, diisopropyl ether, diethyl ether, toluene, chlorobenzene, acetonitrile, and the like or mixtures thereof. Preferred is tetrahydrofuran (THF) and toluene, and most preferred is THF.

A sufficient period of time for stirring at room temperature (20-25° C.) is about 12 hours. However, a shorter period of time may be used, e.g., from about 8 to about 10 hours or a longer period of time, e.g., to upward of 24 hours.

If desired, the sulfinyl group may be cleaved from the diastereomerically pure (S_{S},R)-2-substituted piperidine of Formula (IIIa) to yield the corresponding 2-substituted piperidine enantiomer. A mild acid may be used to achieve removal of the sulfinyl group. Examples of mild acids include, but are not limited to, HCl, trifluoroacetic acid and sulfuric acid. Preferably, HCl is used. Most preferably, 4N HCl in dioxane and MeOH is used for deprotection.

In a further embodiment, there is provided a method for preparing a diastereomerically pure (S_{S},R)-2-substituted piperidine of Formula (IIIa) as set forth above comprising reductive cyclization of an (S₃)-γ-chloro-N-tert-butanesulfinyl ketimine of Formula IV as set forth above in the solvent THF with the reducing agent lithiumtriethylborohydride (LiBHEt₃) or L-Selectride, at a temperature of about −78° C. for a period of about 3 hours, followed by warming to room temperature and stirring for a sufficient period of time. In both Formula (IIIa) and (IV), R is as defined above.

In another embodiment of the present invention, there is provided a method for preparing a diastereomerically pure (S_{S},S)-2-substituted piperidine of Formula (IIIb):

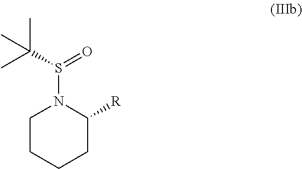

comprising reductive cyclization of (S_{S})-δ-chloro-N-tert-butanesulfinyl ketimine of Formula (IV)

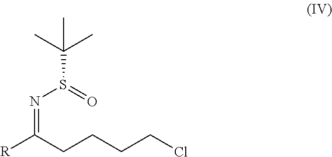

in a suitable solvent with the reducing agent diisobutylaluminum-hydride (DIBAL-H) in the presence of a strong base selected from the group consisting of lithium hexamethyldisilazide (LiHMDS), butyllithium (BuLi), sodium hydride (NaH), triethylamine (CH₂CH₃)₃N or NEt₃) and potassium hydroxide (KOH), wherein the suitable solvent is selected from the group consisting of:
(i) toluene or tetrahydrofuran when the strong base is LiHMDS,
(ii) THF when the strong base is BuLi or NaH, and
(iii) acetonitrile when the strong base is NEt₃, and
(iv) THF—H₂O when the strong base is KOH,
   and at a temperature of about ⁻78 to 0° C. for a period of about 3 hours to 12 hours, followed by warming to room temperature and stirring for a sufficient period of time. In both Formula (IIIb) and (IV), R is the same and is one of alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, or sulfoxide.

Preferably R is a phenyl group.

The R group of both Formula (IIIb) and (IV) may be further substituted with a substituent selected from the group consisting of substituted alkyl; unsubstituted alkyl; substituted alkenyl; unsubstituted alkenyl; substituted alkenyl; unsubstituted alkenyl; alkynyl; alkenylhalogens; hydroxyls; alkoxy, alkenoxy; cycloalkoxy; aryloxy; aralkyloxy; heterocyclyloxy; heterocyclylalkoxy; carbonyls (oxo); carboxyls; urethanes; oximes; hydroxylamines; alkoxyamines; aralkoxyamines; thiols; sulfides; sulfoxides; sulfonamides; amines; N-oxides; hydrazines; hydrazides; hydrazones; azides; amides; ureas; amidines; guanidines; enamines; imides; and nitro groups.

The strong base used in the method of the present invention is selected from the group consisting of lithium hexamethyldisilazide (LiHMDS), butyllithium (BuLi), sodium hydride (NaH), triethylamine (CH₂CH₃)₃N or NEt₃) and potassium hydroxide (KOH). The preferred strong base is lithium hexamethyldisilazide (LiHMDS).

The method of the present invention employs the reducing agent DIBAL-H and the strong base as defined herein in an amount that is at least 1 molar equivalent of the DIBAL-H and at least one molar equivalent of the strong base as defined herein as compared to the (S_{S})-δ-chloro-N-tert-butanesulfinyl ketimine. It is understood that the reducing agent DIBAL-H and the strong base as defined herein may be present in an amount greater than 1 molar equivalent of the (S_{S})-δ-chloro-N-tert-butanesulfinyl ketimine.

The suitable solvent used in the method of the present invention is selected from the group consisting of:
(i) toluene or tetrahydrofuran when the strong base is LiHMDS,
(ii) THF when the strong base is BuLi or NaH, and
(iii) acetonitrile when the strong base is NEt₃, and
(iv) THF—H₂O when the strong base is KOH.
   The preferred solvent is toluene when the strong base is LiHMDS.

A sufficient period of time for stirring at room temperature (20-25° C.) is about 12 hours. However, a shorter period of time may also be sufficient, e.g., from about 8 to about 11 hours or a longer period of time, e.g., to upward of 24 hours.

If desired, the sulfinyl group may be cleaved from the diastereomerically pure (S_{S},S)-2-substituted piperidine of Formula (IIIb) to yield the corresponding 2-substituted piperidine enantiomer. A mild acid may be used to achieve removal of the sulfinyl group. Examples of mild acids include, but are not limited to, HCl, trifluoroacetic acid, and sulfuric acid. Preferably, HCl is used. Most preferably, 4N HCl in dioxane and MeOH is used for deprotection.

In a further embodiment, there is provided a method for preparing a diastereomerically pure (S_{S},S)-2-substituted piperidine of Formula (IIIb) as set forth above comprising reductive cyclization of (S_{S})-δ-chloro-N-tert-butanesulfinyl ketimines of Formula (IV) as set forth above in the suitable solvent toluene with the reducing agent diisobutylaluminum-hydride (DIBAL-H) in the presence of a strong base LiHMDS and at a temperature of about ⁻78° C. for a period of about 3 hours, followed by warming to room temperature and stirring for a sufficient period of time.

It is further understood that the present invention provides a method for preparing a diastereomerically pure (S_{R},S)—N-tert-butanesulfinyl-2-substituted piperadines and (S_{R},R)—N-tert-butanesulfinyl-2-substituted piperadines comprising reductive cyclization of (S_{R})-δ-chloro-N-tert-butanesulfinyl ketimines by applying the same process as set forth above.

In one embodiment of the present invention, there is provided a method for preparing a diastereomerically pure (S_{R},S)-2-substituted piperidine of Formula (IIIc):

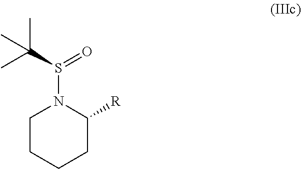

comprising reductive cyclization of (S_{R})-δ-chloro-N-tert-butanesuffinyl ketimine of Formula (IVa)

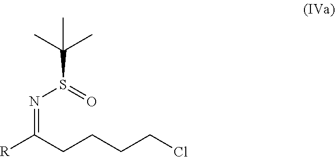

in a suitable solvent with a reducing agent selected from the group consisting of lithiumtriethylborohydride (LiBHEt₃) or L-Selectride at a temperature of about ⁻78 to 23° C. for about 3 hours to 12 hours, followed by warming to about room temperature and stirring for a sufficient period of time. In both Formula (IIIc) and (IVa), R is the same and one of: alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, or sulfoxide.

Preferably R is a phenyl group.

The R group of Formula (IIIc) or (IVa) may be further substituted with a substituent selected from the group consisting of substituted alkyl, unsubstituted alkyl, substituted alkenyl, unsubstituted alkenyl, substituted alkenyl, unsubstituted alkenyl, alkynyl, alkenylhalogens, hydroxyls, alkoxy, alkenoxy, cycloalkoxy, aryloxy, aralkyloxy, heterocyclyloxy, heterocyclylalkoxy, carbonyls (oxo), carboxyls, urethanes, oximes, hydroxylamines, alkoxyamines, aralkoxyamines, thiols, sulfides, sulfoxides, sulfonamides, amines, N-oxides, hydrazines, hydrazides, hydrazones, azides, amides, ureas, amidines, guanidines, enamines, imides, and nitro groups.

The method of the present invention employs the reducing agent LiBHEt₃ or L-Selectride in an amount that is at least 1 molar equivalent of the (S_{R})-δ-chloro-N-tert-butanesulfinyl ketimine. It is understood that the reducing agent LiBHEt₃ or L-Selectride may be present in an amount greater than 1 molar equivalent of the (S_{R})-δ-chloro-N-tert-butanesulfinyl ketimine.

Examples of suitable solvents that may be used with the reducing agents lithiumtriethylborohydride (LiBHEt₃) or L-Selectride in accordance with the present invention include dichloromethane, dichloroethane, chloroform, carbon tetrachloride, tetrahydrofuran (THF), methyl tertbutyl ether, diisopropyl ether, diethyl ether, toluene, chlorobenzene, acetonitrile, and the like or mixtures thereof. Preferred is tetrahydrofuran (THF) and toluene, and most preferred is THF.

A sufficient period of time for stirring at room temperature (20-25° C.) is about 12 hours. However, a shorter period of time may be used, e.g., from about 8 to about 10 hours or a longer period of time, e.g., to upward of 24 hours.

If desired, the sulfinyl group may be cleaved from the diastereomerically pure (S_{R},S)-2-substituted piperidine of Formula (IIIc) to yield the corresponding 2-substituted piperidine enantiomer. A mild acid may be used to achieve removal of the sulfinyl group. Examples of mild acids include, but are not limited to, HCl, trifluoroacetic acid and sulfuric acid. Preferably, HCl is used. Most preferably, 4N HCl in dioxane and MeOH is used for deprotection.

In a further embodiment, there is provided a method for preparing a diastereomerically pure (S_{R},S)-2-substituted piperidine of Formula (IIIc) as set forth above comprising reductive cyclization of an (S_{S})-δ-chloro-N-Pert-butanesulfinyl ketimine of Formula (IVa) as set forth above in the solvent THF with the reducing agent lithiumtriethylborohydride (LiBHEt₃) or L-Selectride, at a temperature of about −78° C. for a period of about 3 hours, followed by warming to room temperature and stirring for a sufficient period of time. In both Formula (IIIc) and Formula (IVa), R is as defined above.

In another embodiment of the present invention, there is provided a method for preparing a diastereomerically pure (S_{R},R)-2-substituted piperidine of Formula (IIId):

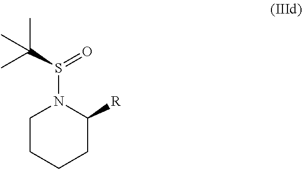

comprising reductive cyclization of (S_{S})-δ-chloro-N-tert-butanesulfinyl ketimine of Formula IVa

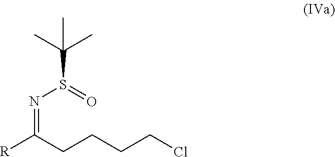

in a suitable solvent with the reducing agent diisobutylaluminum-hydride (DIBAL-H) in the presence of a strong base selected from the group consisting of lithium hexamethyldisilazide (LiHMDS), butyllithium (BuLi), sodium hydride (NaH), triethylamine (CH₂CH₃)₃N or NEt₃) and potassium hydroxide (KOH), wherein the suitable solvent is selected from the group consisting of:
(i) toluene or tetrahydrofuran when the strong base is LiHMDS,
(ii) THF when the strong base is BuLi or NaH, and
(iii) acetonitrile when the strong base is NEt₃, and
(iv) THF—H₂O when the strong base is KOH,
   and at a temperature of about ⁻78 to 0° C. for a period of about 3 hours to 12 hours, followed by warming to room temperature and stirring for a sufficient period of time. In both Formula (IIId) and (IVa), R is the same and is one of alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, or sulfoxide.

Preferably R is a phenyl group.

The R group of both Formula (IIId) and (IVa) may be further substituted with a substituent selected from the group consisting of substituted alkyl; unsubstituted alkyl; substituted alkenyl; unsubstituted alkenyl; substituted alkenyl; unsubstituted alkenyl; alkynyl; alkenylhalogens; hydroxyls; alkoxy, alkenoxy; cycloalkoxy; aryloxy; aralkyloxy; heterocyclyloxy; heterocyclylalkoxy; carbonyls (oxo); carboxyls; urethanes; oximes; hydroxylamines; alkoxyamines; aralkoxyamines; thiols; sulfides; sulfoxides; sulfonamides; amines; N-oxides; hydrazines; hydrazides; hydrazones; azides; amides; ureas; amidines; guanidines; enamines; imides; and nitro groups.

The strong base used in the method of the present invention is selected from the group consisting of lithium hexamethyldisilazide (LiHMDS), butyllithium (BuLi), sodium hydride (NaH), triethylamine (CH₂CH₃)₃N or NEt₃) and potassium hydroxide (KOH). The preferred strong base is lithium hexamethyldisilazide (LiHMDS).

The method of the present invention employs the reducing agent DIBAL-H and the strong base as defined herein in an amount that is at least 1 molar equivalent of the DIBAL-H and at least one molar equivalent of the strong base as defined herein as compared to the (S_{S})-δ-chloro-N-tert-butanesulfinyl ketimine. It is understood that the reducing agent DIBAL-H and the strong base as defined herein may be present in an amount greater than 1 molar equivalent of the (S_{S})-δ-chloro-N-tert-butanesulfinyl ketimine.

The suitable solvent used in the method of the present invention is selected from the group consisting of:
(i) toluene or tetrahydrofuran when the strong base is LiHMDS,
(ii) THF when the strong base is BuLi or NaH, and
(iii) acetonitrile when the strong base is NEt₃, and
(iv) THF—H₂O when the strong base is KOH.
   The preferred solvent is toluene when the strong base is LiHMDS.

A sufficient period of time for stirring at room temperature (20-25° C.) is about 12 hours. However, a shorter period of time may also be sufficient, e.g., from about 8 to about 11 hours or a longer period of time, e.g., to upward of 24 hours.

If desired, the sulfinyl group may be cleaved from the diastereomerically pure (S_{R},R)-2-substituted piperidine of Formula (IIId) to yield the corresponding 2-substituted piperidine enantiomer. A mild acid may be used to achieve removal of the sulfinyl group. Examples of mild acids include, but are not limited to, HCl, trifluoroacetic acid, and sulfuric acid. Preferably, HCl is used. Most preferably, 4N HCl in dioxane and MeOH is used for deprotection.

In a further embodiment, there is provided a method for preparing a diastereomerically pure (S_{R},R)-2-substituted piperidine of Formula (IIId) as set forth above comprising reductive cyclization of (S_{SR})-δ-chloro-N-tert-butanesulfinyl ketimines of Formula (IVa) as set forth above in the suitable solvent toluene with the reducing agent diisobutylaluminum-hydride (DIBAL-H) in the presence of a strong base LiHMDS and at a temperature of about ⁻78° C. for a period of about 3 hours, followed by warming to room temperature and stirring for a sufficient period of time.

During synthesis of the (S_{S},R)-2-substituted pyrrolidines, (S_{R},S)-2-substituted pyrrolidines, (S_{S},R)-2-substituted piperidines and (S_{R},S)-2-substituted piperidines of the present invention, the reactants are initially contacted in accordance with the present technology at about −78 to 23° C., and preferably at about −78° C. The reactants are contacted at the temperature of about −78 to 23° C. in accordance with the present technology for a period of about 3 hours to 12 hours, and preferably for about 3 hours.

During synthesis of the (S_{S},S)-2-substituted pyrrolidines, (S_{R},R)-2-substituted pyrrolidines, (S_{S},S)-2-substituted piperidines and (S_{R},R)-2-substituted piperidines of the present invention, the reactants are contacted in accordance with the present technology at about −78° C. to about 0° C., and preferably at about −78° C. The reactants are initially contacted at the temperature of about −78° C. to about 0° C. in accordance with the present technology for a period of about 3 hours to 12 hours, and preferably for about 3 hours.

The method of the present invention is used to prepare a diastereomerically pure diastereomer of the 2-substituted pyrrolidine of Formulas (Ia) to (Id) as set forth above or diastereomer of the 2-substituted piperadines of Formulas (IIIa) to (IIId) as set forth above. As referred to herein, the term “diastereomerically pure” shall refer to a resulting 2-substituted pyrrolidine or 2-substituted piperadine product prepared in accordance with the present invention wherein the diastereomeric ratio is ranging between about 95:5 to 99:1, preferably ranging between 98:2 to 99:1, and most preferably 99:1.

The following terms are used throughout as defined below.

In general, “substituted” refers to an organic group as defined below (e.g., an alkyl group or aryl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, in accordance with the present invention, various aromatic and aliphatic substituents may be substituted at the 2 position on the pyrrolidine or piperidine ring.

A substituted group may be further substituted with one or more substituents. In some embodiments, a substituted group is substituted with 1, 2, 3, 4, 5, or 6 substituents. Examples of substituent groups include halogens (i.e., F, Cl, Br, and I); hydroxyls; alkoxy, alkenoxy, cycloalkoxy, aryloxy, aralkyloxy, heterocyclyloxy, and heterocyclylalkoxy groups; carbonyls (oxo); carboxyls; urethanes; oximes; hydroxylamines; alkoxyamines; aralkoxyamines; thiols; sulfides; sulfoxides; sulfonamides; amines; N-oxides; hydrazines; hydrazides; hydrazones; azides; amides; ureas; amidines; guanidines; enamines; imides; nitro groups; and the like.

Substituted ring groups such as substituted cycloalkyl, cycloalkenyl, aryl, heterocyclyl and heteroaryl groups also include rings and fused ring systems in which a bond to a hydrogen atom is replaced with a bond to a carbon atom. Therefore, substituted cycloalkyl, cycloalkenyl, aryl, heterocyclyl and heteroaryl groups may also be substituted with: substituted or unsubstituted alkyl, alkenyl, and alkynyl groups as defined below.

As used herein, the term “alkyl” refers to straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, carboxyalkyl, and the like.

As used herein, the term “cycloalkyl” refers to mono-, bi- or tricyclic alkyl groups having from 3 to 14 carbon atoms in the ring(s), or, in some embodiments, 3 to 12, 3 to 10, 3 to 8, or 3, 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups as described below, and fused rings, such as, but not limited to, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above. For example, substituted phenyl compounds can comprise different mono substituted, different di substituted different tri substituted, different tetra substituted, and different penta rings substituted with fluoro, chloro, bromo, trifluoro, hydroxy, methoxy, ethoxy, propoxy, acetoxy, benzoxy, methyl, ethyl, propyl etc.

As used herein, the term “bridged cycloalkyl” refers to cycloalkyl groups in which two or more hydrogen atoms on the same or different carbon atoms are replaced by an alkylene bridge, wherein the bridge can contain 1 to 6 carbon atoms Bridged cycloalkyl groups can be bicyclic, such as, for example bicyclo[2.1.1]hexane, or tricyclic, such as, for example, adamantyl. Representative bridged cycloalkyl groups include bicyclo[2.1.1]hexyl, bicyclo[2.2.1]heptyl, bicyclo[3.2.1]octyl, bicyclo[2.2.2]octyl, bicyclo[3.2.2]nonyl, bicyclo[3.3.1]nonyl, bicyclo[3.3.2]decanyl, adamantyl, noradamantyl, bornyl, or norbornyl groups. Substituted bridged cycloalkyl groups may be substituted one or more times with non-hydrogen and non-carbon groups as defined above. Representative substituted bridged cycloalkyl groups may be mono-substituted or substituted more than once, such as, but not limited to, mono-, di- or tri-substituted adamantyl groups, which may be substituted with substituents such as those listed above.

As used herein, the term “cycloalkylalkyl” refers to alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

As used herein, the term “alkenyl” refers to straight and branched chain alkyl groups as defined above, except that at least one double bond exists between two carbon atoms. Thus, alkenyl groups have from 2 to 12 carbon atoms, and typically from 2 to 10 carbons or, in some embodiments, from 2 to 8, 2 to 6, or 2 to 4 carbon atoms. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

As used herein, the term “cycloalkenyl” refers to cycloalkyl groups as defined above, having at least one double bond between two carbon atoms. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, butadienyl, pentadienyl, and hexadienyl.

As used herein, the term “cycloalkenylalkyl” refers to alkyl groups as defined above in which a hydrogen or carbon bond of the alkyl group is replaced with a bond to a cycloalkenyl group as defined above. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

As used herein, the term “alkynyl” refers to straight and branched chain alkyl groups as defined above, except that at least one triple bond exists between two carbon atoms. Thus, alkenyl groups have from 2 to 12 carbon atoms, and typically from 2 to 10 carbons or, in some embodiments, from 2 to 8, 2 to 6, or 2 to 4 carbon atoms. Examples of alkynl groups include ethynyl, propynyl, n-butynyl, i-butynyl, 3-methylbut-2-ynyl, and n-pentynyl, among others. Representative substituted alkynyl groups may be mono-substituted or substituted more than once, such as, but not limited to, mono-, di- or tri-substituted with substituents such as those listed above.

As used herein, the term “aryl” refers to cyclic aromatic hydrocarbons that do not contain heteroatoms. Aryl groups herein include monocyclic, bicyclic and tricyclic ring systems. Thus, aryl groups include, but are not limited to, phenyl (or “Ph”), azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to herein as “substituted aryl groups”. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

As used herein, the term “aralkyl” or “arylalkyl” refers to alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

As used herein, the term “heterocyclyl” refers to aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to herein as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl(pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl(azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

As used herein, the term “heteroaryl” refers to aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl(pyrrolopyridinyl), indazolyl, benzimidazolyl, imidazopyridinyl(azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

As used herein, the term “heterocyclylalkyl” refers to alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

As used herein, the term “heteroaralkyl” refers to alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

As used herein, the term “halo” and “halogen” refers to fluoro, chloro, bromo, or iodo. Preferred are fluoro, chloro or bromo, and more preferred are fluoro or chloro.

As used herein, the term “haloalkyl” refers to an alkyl group as defined herein substituted with one or more halogen atoms. Representative haoloalkyl groups include chloromethyl, bromoethyl, trifluoromethyl, and the like.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

As used herein, the term “alkoxy” refers to hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Representative substituted alkoxy groups may be substituted one or more times with substituents such as those listed above.

As used herein, the term “alkenoxy” refers to hydroxyl groups (—OH) in which the bond to the hydrogen atom is replaced by a bond to a carbon atom of a substituted or unsubstituted alkenyl group as defined above. Representative substituted alkenoxy groups may be substituted one or more times with substituents such as those listed above.

As used herein, the term “cycloalkoxy” refers to an cycloalkyl-O— group in which the cycloalkyl group is as previously described. Examples of cycloalkoxy groups include but are not limited to cyclopropyloxy, cyclobutyloxy, cyclopentyloxy, cyclohexyloxy, and the like. Representative substituted cycloalkoxy groups may be substituted one or more times with substituents such as those listed above.

As used herein, the terms “aryloxy” and “aralkyloxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

As used herein, the term “heterocyclyloxy” refers to a heterocyclyl group attached to the parent molecular moiety through an oxygen atom. Representative substituted heterocyclyloxy groups may be substituted one or more times with substituents such as those listed above.

As used herein, the term “heterocyclylalkoxy” refers to a heterocyclyl group attached to the parent molecular moiety through an alkoxy group. Representative substituted heterocyclylalkoxy groups may be substituted one or more times with substituents such as those listed above.

As used herein, the term “amide” (or “amido”) refers to C- and N-amide groups, i.e., —C(O)NRR, and —NRC(O)R groups, respectively. The R groups are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H).

As used herein, the term “urethane” refers to N- and O-urethane groups, i.e., —NC(O)OR and —OC(O)NRR groups, respectively. The R groups are independently unsubstituted or unsubstituted alkyl, alkenyl, alkynyl, cycloalkyl, aryl, aralkyl, heterocyclylalkyl, or heterocyclyl group as defined herein. R may also be H.

As used herein, the term “amine” (or “amino”) refers to —NHR and —NRR groups, wherein the R groups are independently hydrogen, or a substituted or unsubstituted alkyl, alkenyl, alkynyl, cycloalkyl, aryl, aralkyl, heterocyclylalkyl or heterocyclyl group as defined herein. In some embodiments, amino is NH₂, methylamino, dimethylamino, ethylamino, diethylamino, propylamino, isopropylamino, phenylamino, or benzylamino.

As used herein, the terms sulfonamido” and “sulfonamide” refer to S- and N-sulfonamide groups, i.e., —SO₂NRR and —NRSO₂R groups, respectively. R groups are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂).

As used herein, the term “thiol” refers to —SH groups, while sulfides include —SR groups, and sulfoxides include —S(O)R groups, wherein the R groups are each independently a substituted or unsubstituted alkyl, cycloalkyl, alkenyl, alkynyl, aryl aralkyl, heterocyclyl or heterocyclylalkyl group as defined herein.

As used herein, the term “urea” refers to —NR—C(O)—NRR groups wherein the R groups are independently hydrogen, or a substituted or unsubstituted alkyl, alkenyl, alkynyl, cycloalkyl, aryl, aralkyl, heterocyclyl, or heterocyclylalkyl group as defined herein.

As used herein, the term “amidine” refers to —C(NR)NRR and —NRC(NR)R, wherein the R groups are each independently hydrogen, or a substituted or unsubstituted alkyl, cycloalkyl, alkenyl, alkynyl, aryl aralkyl, heterocyclyl or heterocyclylalkyl group as defined herein.

As used herein, the term “guanidine” refers to —NRC(NR)NRR, wherein the R groups are each independently hydrogen, or a substituted or unsubstituted alkyl, cycloalkyl, alkenyl, alkynyl, aryl aralkyl, heterocyclyl or heterocyclylalkyl group as defined herein.

As used herein, the term “enamine” refers to —C(R)═C(R)NRR and —NRC(R)═C(R)R, wherein the R groups are each independently hydrogen, a substituted or unsubstituted alkyl, cycloalkyl, alkenyl, alkynyl, aryl aralkyl, heterocyclyl or heterocyclylalkyl group as defined herein.

As used herein, the term “imide” refers to —C(O)NRC(O)R, wherein the R groups are each independently hydrogen, or a substituted or unsubstituted alkyl, cycloalkyl, alkenyl, alkynyl, aryl aralkyl, heterocyclyl or heterocyclylalkyl group as defined herein.

As used herein, the term “protected” with respect to amino groups refers to forms of these functionalities which are protected from undesirable reaction by means of protecting groups. Protecting groups are known to those skilled in the art and can be identified, added or removed using well-known procedures such as those set forth in Protective Groups in Organic Synthesis, McOmie, Plenum Press, London, N.Y. (1973) and Protective Groups in Organic Synthesis, Greene, T. W.; Wuts, P. G. M., John Wiley & Sons, New York, N.Y., (3rd Edition, 1999). Examples of nitrogen protecting groups include, but are not limited to, substituted or unsubstituted sulfinyl groups.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

The following examples further illustrate the invention and are not meant in any way to limit the scope thereof.

### Example 1

### Synthesis of γ-chlorinated N-tert-butanesulfinyl ketimines (S_{S})-1a-1p

Existing methodologies are used for the synthesis of γ-chlorinated N-tert-butanesulfinyl ketimines (S_{S})-1a-1l by condensation of appropriate ketones 4a-4l with (S)-tert-butanesulfinamide 7 (Scheme 2). (See, for example, Ellman et al, Accounts of Chemical Research, 35, 984-995 (2002).) A trial using homogenous conditions that utilize Ti(OEt)₄ as both a Lewis acid and a water scavenger furnishes 1a in 90% yield. Since this approach is straightforward and provides 1a with excellent yields, it is employed to prepare aryl sulfinyl ketimine 1a-1i, heteroaryl sulfinyl ketimine 1j and aliphatic sulfinyl ketimines 1k-1l.

In the same way, the δ-chlorinated N-tert-butanesulfinyl ketimine (S_{S})-1m, ε-chlorinated N-tert-butanesulfinyl ketimine (S_{S})-1n, ζ-chlorinated N-tert-butanesulfinyl ketimine (S_{S})-1o, and dechlorinated N-tert-butanesulfinyl ketimine (S_{S})-1p is synthesized via condensation of (S)-tert-butanesulfinamide 5 with ketones 4m-4p, respectively. In all cases, the products are isolated in analytically pure form by extractive workup followed by flash chromatography. ¹H NMR analysis reveals that sulfinyl ketimines 1a-1l existed solely as the (E)-isomer, and the corresponding (Z)-sulfinyl ketimines were not observed

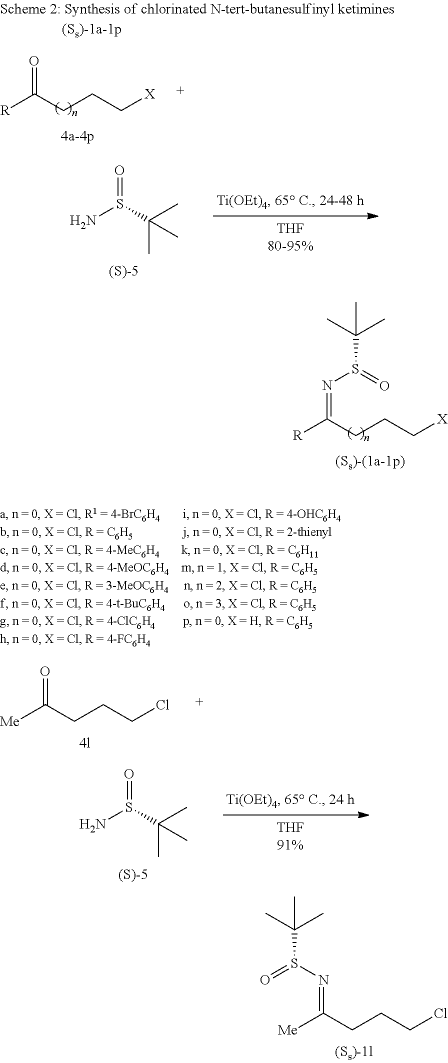

### General Procedure (GP1) for the Synthesis of γ-Chloro N-Sulfinyl Ketimine 1

A 500 mL, three-necked, round-bottomed flask is charged with 1-(4-bromophenyl)-4-chlorobutan-1-one 4a (40.0 mmol), THF (100 mL), tert-butanesulfinamide 5 (60.0 mmol), and Ti(OEt)₄ (80.0 mmol) under nitrogen atmospheres. The reaction mixture is then refluxed at 65° C. for 48 hours. After completion, the reaction is allowed to cool to room temperature. Isopropyl acetate (100 mL) and saturated NaCl solution (100 mL) is then added to this mixture and stirred for 1 hour. The solids are removed by filtration and filtrate is washed with water (2×50 mL). The organic phase is evaporated under vacuum to dryness to obtained crude product. The crude product is purified by flash column chromatography (silica gel, 10% ethyl acetate in heptanes) to afford the pure γ-Chloro N-sulfinyl ketimine 1a.

### (S,E)-N-(4-chloro-1-(4-bromophenyl)butylidene)-2-methylpropane-2-sulfinamide (1a)

Following the general procedure (GP1), the reaction of 4-chloro-1-(4-bromophenyl)-butan-1-one (4a) (25.5 g, 100.0 mmol) with (S_{S})-tert-butanesulfinamide (13.3 g, 110.0 mmol) and Ti(OEt)₄ (34.5 g, 150.0 mmol) yields 30.6 g (84%) of pure γ-Chloro N-sulfinyl ketimine (S_{S}) 1a as white solid, mp=48-50° C., [α]²⁵_{D}=−24.18°, (c 1.10, MeOH). ¹H NMR (501 MHz, CHLOROFORM-d) δ ppm 1.24-1.42 (m, 9H) 2.01-2.28 (m, 2H) 3.19-3.51 (m, 2H) 3.57-3.70 (m, 2H) 7.56 (d, J=8.51 Hz, 2H) 7.66-7.86 (m, 2H). ¹³C NMR (125 MHz, CHLOROFORM-d) δ ppm 176.8, 136.3, 131.9, 128.9, 126.5, 58.0, 44.5, 31.5, 29.8, 22.7. HRMS (EI) Calculated for C₁₄H₂₀NOSBrCl [M+H]: 364.0120 Found 364.0138.

### (S,E)-N-(4-chloro-1-phenyl)butylidene)--2-methylpropane-2-sulfinamide (1b)

Following the general procedure (GP1), the reaction of 4-chloro-1-phenyl-butan-1-one (4b) (18.2 g, 100.0 mmol) with (S_{S})-tert-butanesulfinamide (13.3 g, 110.0 mmol) and Ti(OEt)₄ (34.5 g, 150.0 mmol) yields 25.9 g (91%) of pure γ-Chloro N-sulfinyl ketimine (S_{S}) 1b as white solid, mp=35-36° C., [α]²⁵_{D}=−20.90°, (c 1.10, MeOH). ¹H NMR (501 MHz, CHLOROFORM-d) δ ppm 1.36 (s, 9H) 2.07-2.28 (m, 2H) 3.23-3.54 (m, 2H) 3.57-3.72 (m, 2H) 7.33-7.56 (m, 3H) 7.79-7.96 (m, 2H). ¹³C NMR (125 MHz, CHLOROFORM-d) δ ppm 178.0, 137.5, 131.7, 128.7, 127.4, 57.9, 44.6, 31.6, 30.0, 22.7. HRMS (EI) Calculated for C₁₄H₂₁NOSI [M+H]: 286.1024. Found 286.1032

### (S,E)-N-(4-chloro-1-p-tolylbutylidene)-2-methylpropane-2-sulfinamide (1c)

Following the general procedure (GP1), the reaction of 4-chloro-1-p-tolyl-butan-1-one (4c) (25 g, 127.0 mmol) with (S_{S})-tert-butanesulfinamide (23.1 g, 190.6 mmol) and Ti(OEt)₄ (57.9 g, 254.2 mmol) yields 34.3 g (90%) of pure γ-Chloro N-sulfinyl ketimine (S_{S}) 1c as viscous oil [α]²⁵_{D}=−17.46°, (c 1.03, MeOH). ¹H NMR (501 MHz, CHLOROFORM-d) δ ppm 1.34 (s, 9H) 2.08-2.25 (m, 2H) 2.40 (s, 3H) 3.22-3.49 (m, 2H) 3.59-3.67 (m, 2H) 7.24 (d, J=8.20 Hz, 2H) 7.74-7.83 (m, 2 H). ¹³C NMR (125 MHz, CHLOROFORM-d) δ ppm 178.0, 142.4, 134.8, 129.4, 127.5, 57.7, 53.4, 44.7, 31.7, 30.0, 22.7, 21.4. HRMS (EI) Calculated for C₁₅H₂₃NOSCl [M+H]: 300.1189. Found 300.1176.

### (S,E)-N-(4-chloro-1-(4-methoxyphenyl)butylidene)-2-methylpropane-2-sulfinamide (1d)

Following the general procedure (GP1), the reaction of 4-chloro-1-(4-methoxyphenyl)butan-1-one (4d) (25 g, 117.5 mmol) with (S_{S})-tert-butanesulfinamide (21.37 g, 176.32 mmol) and Ti(OEt)₄ (57.9 g, 235.1 mmol) yields 31.56 g (85%) of pure γ-Chloro N-sulfinyl ketimine (S_{S}) 1d as viscous oil [α]²⁵_{D}=−28.26°, (c 1.15, MeOH). ¹H NMR (501 MHz, CHLOROFORM-d) δ ppm 1.32 (s, 9H) 2.07-2.27 (m, 2H) 3.19-3.47 (m, 2H) 3.59-3.68 (m, 2H) 3.86 (s, 3H) 6.88-6.97 (m, 2H) 7.83-7.91 (m, 2H). ¹³C NMR (125 MHz, CHLOROFORM-d) δ ppm 178.0, 162.6, 130.0, 129.4, 114.4, 57.5, 55.4, 53.4, 44.8, 31.7, 29.9, 22.6. HRMS (EI) Calculated for C₁₅H₂₃NO₂SCl [M+H]: 316.1138. Found 316.1125.

### (S,E)-N-(1-(4-tert-butylphenyl)-4-chlorobutylidene)-2-methylpropane-2-sulfinamide (1e)

Following the general procedure (GP1), the reaction of 1-(4-tert-butylphenyl)-4-chlorobutan-1-one (4e) (10 g, 41.885 mmol) with (S_{S})-tert-butanesulfinamide (7.614 g, 62.83 mmol) and Ti(OEt)₄ (19.112 g, 83.77 mmol) yields 13.6 g (90%) of pure γ-Chloro N-sulfinyl ketimine (S_{S}) 1e as viscous oil [α]²⁵_{D}=−18.9°, (c 1.0, MeOH). ¹H NMR (501 MHz, CHLOROFORM-d) δ ppm 7.76-7.90 (m, 2H), 7.45 (d, J=7.57 Hz, 2H), 3.59-3.69 (m, 2H), 3.21-3.51 (m, 2H), 2.08-2.27 (m, 2H), 1.34 (s, 18H). ¹³C NMR (125 MHz, CHLOROFORM-d) δ ppm 177.8, 155.3, 134.7, 127.3, 125.6, 57.7, 53.4, 44.7, 34.9, 31.7, 31.1, 28.1, 22.7. HRMS (EI) Calculated for C₁₈H₂₉NOSCl [M+H]: 342.1658. Found 342.1624.

### (S,E)-N-(4-chloro-1-(4-hydroxyphenyl)butylidene)-2-methylpropane-2-sulfinamide (1f)

Following the general procedure (GP1), the reaction of 5-chloro-1-(4-hydroxy-phenyl)butan-1-one (4f) (10 g, 50.34 mmol) with (S_{S})-tert-butanesulfinamide (9.15 g, 75.51 mmol) and Ti(OEt)₄ (22.969 g, 100.68 mmol) yields 9.87 g (65%) of pure γ-Chloro N-sulfinyl ketimine (S_{S}) 1f as a solid, mp=88-90° C., [α]²⁵_{D}=−52.05°, (c 1.04, MeOH). ¹H NMR (501 MHz, DMSO-d₆) δ ppm 1.21 (s, 9H) 1.85-2.12 (m, 2H) 3.10-3.37 (m, 2H) 3.71 (t, J=6.46 Hz, 2H) 6.85 (d, J=8.83 Hz, 2H) 7.80 (d, J=5.67 Hz, 2H) 10.22 (s, 1H). ¹³C NMR (125 MHz, DMSO-d₆)) δ ppm 178.0, 161.1, 129.6, 127.7, 115.4, 56.4, 44.7, 31.4, 29.4 22.0. HRMS (EI) Calculated for C₁₄H₂₁NO₂SCl [M+H]: 302.0982. Found 302.1011.

### (S,E)-N-(4-chloro-1-(3-methoxyphenyl)butylidene)-2-methylpropane-2-sulfinamide (1g)

Following the general procedure (GP1), the reaction of 5-chloro-1-(3-methoxyphenyl)butan-1-one (4g) (5 g, 23.51 mmol) with (S_{S})tert-butanesulfinamide (4.274 g, 35.26 mmol) and Ti(OEt)₄ (10.72 g, 47.02 mmol) yields 6.08 g (82%) of pure γ-Chloro N-sulfinyl ketimine (S_{S}) 1g as a viscous liquid. [α]²⁵_{D}=−10.42°, (c 1.42, MeOH). ¹H NMR (501 MHz, CHLOROFORM-d) δ ppm 7.40-7.49 (m, 2H), 7.34 (t, J=7.88 Hz, 1H), 7.03 (d, J=7.25 Hz, 1H), 3.83 (s, 3H), 3.60-3.66 (m, 2H), 3.23-3.48 (m, 2H), 2.08-2.25 (m, 2H), 1.33 (s, 9H). ¹³C NMR (125 MHz, CHLOROFORM-d) δ ppm 177.7, 159.7, 138.9, 129.6, 119.8, 117.5, 112.6, 57.9, 55.3, 44.6, 31.7, 30.0, 22.7. HRMS (EI) Calculated for C₁₅H₂₃NO₂SCl [M+H]: 316.1138. Found 316.1104.

### (S,E)-N-(4-chloro-1-(4-chlorophenyl)butylidene)-2-methylpropane-2-sulfinamide (1h)

Following the general procedure (GP1), the reaction of 4-chloro-1-(4-chlorophenyl)butan-1-one (4h) (10 g, 46.064 mmol) with (S_{S})-tert-butanesulfinamide (8.374 g, 69.095 mmol) and Ti(OEt)₄ (21.018 g, 92.127 mmol) yields 13.7 g (93%) of pure γ-Chloro N-sulfinyl ketimine (S_{S}) 1h as a white color solid. mp=40-42° C., [α]²⁵_{D}=−26.96°, (c 1.12, MeOH). ¹H NMR (501 MHz, CHLOROFORM-d) δ ppm 7.75-7.88 (m, 2H), 7.41 (d, J=8.51 Hz, 2H), 3.59-3.69 (m, 2H), 3.23-3.49 (m, 2H), 2.04-2.25 (m, 2H), 1.32 (s, 9H). ¹³C NMR (125 MHz, CHLOROFORM-d) δ ppm 176.8, 138.0, 135.9, 129.0, 128.7, 58.0, 44.6, 31.6, 29.8, 22.7. HRMS (EI) Calculated for C₁₄H₂₀NOSCl₂ [M+H]: 320.0643. Found 320.0683.

### (S,E)-N-(4-chloro-1-(4-fluorophenyl)butylidene)-2-methylpropane-2-sulfinamide (1i)

Following the general procedure (GP1), the reaction of 4-chloro-1-(4-fluorophenyl)butan-1-one (4i) (10 g, 49.84 mmol) with (S_{S})-tert-butanesulfinamide (9.06 g, 74.76 mmol) and Ti(OEt)₄ (22.74 g, 99.68 mmol) yields 14.2 g (94%) of pure γ-Chloro N-sulfinyl ketimine (S_{S}) 1i as a white color solid. mp=38-40° C. [α]²⁵_{D}=−40.76°, (c 1.05, MeOH). ¹H NMR (501 MHz, CHLOROFORM-d) δ ppm 7.85-7.96 (m, 2H), 7.12 (t, J=8.51 Hz, 2H), 3.60-3.70 (m, 2H), 3.23-3.49 (m, 2H), 2.08-2.26 (m, 2H), 1.35 (s, 9H). ¹³C NMR (125 MHz, CHLOROFORM-d) δ ppm 176.8, 165.9, 163.9, 133.7, 129.8, 129.7, 115.8, 115.7, 115.4, 57.8, 44.6, 31.6, 29.9, 22.7. HRMS (EI) Calculated for C₁₄H₂₀NOSClF [M+H]: 304.0938. Found 304.0926.

### (S,E)-N-(4-chloro-1-(thiophen-2-yl)butylidene)-2-methylpropane-2-sulfinamide (1j)

Following the general procedure (GP1), the reaction of 4-chloro-1-(thiophen-2-yl)butan-1-one (4j) (5 g, 26.5 mmol) with (S_{S})-tert-butanesulfinamide (4.817 g, 39.75 mmol) and Ti(OEt)₄ (12.09 g, 53.0 mmol) yields 7.5 g (97%) of pure γ-Chloro N-sulfinyl ketimine (S_{c}) 1j as a viscous liquid. [α]²⁵_{D}=−90.05°, (c 1.06, MeOH). ¹H NMR (501 MHz, CHLOROFORM-d) δ ppm 7.58 (d, J=3.15 Hz, 1H), 7.50 (d, J=5.04 Hz, 1H), 7.07-7.12 (m, 1H), 3.60-3.70 (m, 2H), 3.33-3.42 (m, 1H), 3.23 (dd, J=10.72, 5.67 Hz, 1H), 2.15-2.35 (m, 2H), 1.30 (s, 9H). ¹³C NMR (125 MHz, CHLOROFORM-d) δ ppm 172.3, 144.9, 132.3, 129.6, 128.0, 58.0, 44.6, 32.1, 30.6, 22.5. HRMS (EI) Calculated for C₁₂H₁₉NOS₂Cl [M+H]: 292.0597. Found 292.0573.

### (S,E)-N-(4-chloro-1-cyclohexyl-butylidene)-2-methylpropane-2-sulfinamide (1k)

Following the general procedure (GP1), the reaction of 4-chloro-1-cyclohexyl-butan-1-one (4k) (9.1 g, 50.0 mmol) with (S_{S})-tert-butanesulfinamide (9.06 g, 74.76 mmol) and Ti(OEt)₄ (22.74 g, 99.68 mmol) yields 13.2 g (90%) of pure γ-Chloro N-sulfinyl ketimine (S_{S}) 1k as a viscous oil. [α]²⁵_{D}=−32.82°, (c 1.02, MeOH). ¹H NMR (501 MHz, CHLOROFORM-d) δ ppm 3.53-3.66 (m, 2H) 2.72-2.96 (m, 2H) 2.54-2.65 (m, 1H) 2.20-2.35 (m, 1H) 2.02-2.15 (m, 2H) 1.65-1.91 (m, 4H) 1.10-1.48 (s and m, 14H). ¹³C NMR (125 MHz, CHLOROFORM-d) δ ppm 189.2, 56.8, 49.6, 44.5, 32.6, 30.6, 25.9, 25.8, 22.3, 22.1. HRMS (EI) Calculated for C₁₄H₂₇NOSCl [M+H]: 292.8883. Found 292.8885.

### (S,E)-N-(4-chloro-1-methyl)-2-methylpropane-2-sulfinamide (1l)

Following the general procedure (GP1), the reaction of 4-chloro-1-methyl-butan-1-one (4l) (12.0 g, 100.0 mmol) with (S_{S})-tert-butanesulfinamide (13.3 g, 110.0 mmol) and Ti(OEt)₄ (34.5 g, 150.0 mmol) yields 18.8 g (85%) of pure γ-Chloro N-sulfinyl ketimine (S_{S}) 1l as a viscous liquid, [α]²⁵_{D}=−21.12°, (c 1.60, MeOH). ¹H NMR (501 MHz, CHLOROFORM-d) δ ppm 1.24 (s, 9H) 2.00-2.16 (m, 2H) 2.35 (s, 3H) 2.60 (t, J=7.09 Hz, 2H) 3.60 (t, J=6.31 Hz, 2H). ¹³C NMR (125 MHz, CHLOROFORM-d) δ ppm 183.8, 56.3, 44.2, 39.9, 27.9, 23.3, 22.1. HRMS (EI) Calculated for C₉H₁₉ClNOS [M+H]: 224.0876. Found 224.0876.

### (S,E)-N-(5-chloro-1-phenylpentylidene)-2-methylpropane-2-sulfinamide (1m)

Following the general procedure (GP1), the reaction of 5-chloro-1-phenylpentan-1-one (4m) (10 g, 50.84 mmol) with (S_{S})-tert-butanesulfinamide (9.24 g, 76.26 mmol) and Ti(OEt)₄ (23.2 g, 101.68 mmol) yields 13.72 g (90%) of pure γ-Chloro N-sulfinyl ketimine (S_{S}) 1m as a viscous liquid. [α]²⁵_{D}=+23.61°, (c 1.02, MeOH). ¹H NMR (501 MHz, CHLOROFORM-d) δ ppm 7.78-7.91 (m, 2H), 7.39-7.51 (m, 3H), 3.51-3.61 (m, 2H), 3.14-3.37 (m, 2H), 1.78-1.95 (m, 4H), 1.33 (s, 9 H). ¹³C NMR (125 MHz, CHLOROFORM-d) δ ppm 179.0 137.7, 131.5, 128.6, 127.3, 57.7, 44.3, 32.3, 31.3, 25.9, 22.7. HRMS (EI) Calculated for C₁₅H₂₃NOSCl [M+H]: 300.1189. Found 300.1151.

### (S,E)-N-(6-chloro-1-phenylhexylidene)-2-methylpropane-2-sulfinamide (1n)

Following the general procedure (GP1), the reaction of 6-chloro-1-phenylhexan-1-one (4n) (10 g, 47 mmol) with (S_{S})-tert-butanesulfinamide (8.6 g, 71.4 mmol) and Ti(OEt)₄ (20.71 g, 94 mmol) yields 13.42 g (90%) of pure γ-Chloro N-sulfinyl ketimine (S_{S}) 1n as a viscous liquid. [α]²⁵_{D}=+12.41°, (c 1.22, MeOH). ¹H NMR (501 MHz, CHLOROFORM-d) δ ppm 7.83 (br. s., 2H), 7.40-7.51 (m, 3 H), 3.57 (none, 1H), 3.51 (t, J=6.46 Hz, 2H), 3.23-3.34 (m, 1H), 3.12-3.22 (m, 1H), 1.77-1.85 (m, 2H), 1.65-1.74 (m, 2H), 1.54-1.63 (m, 2H), 1.35 (s, 9H). ¹³C NMR (125 MHz, CHLOROFORM-d) δ ppm 179.5, 137.8, 131.5, 128.6, 127.3, 57.3, 44.7, 32.2, 32.0, 27.9, 27.0, 22.7. HRMS (EI) Calculated for C₁₆H₂₅NOSCl [M+H]: 314.1345. Found 314.1347.

### (S,E)-N-(7-chloro-1-phenylhexylidene)-2-methylpropane-2-sulfinamide (1o)

Following the general procedure (GP1), the reaction of 7-chloro-1-phenylheptan-1-one (4o) (11.5 g, 50.0 mmol) with (S_{S})-tert-butanesulfinamide (9.0 g, 75.0 mmol) and Ti(OEt)₄ (22.8 g, 100.0 mmol) yields 14.5 g (89%) of pure γ-Chloro N-sulfinyl ketimine (S_{S}) 1o as a viscous liquid. [α]²⁵_{D} 29.08°, (c 1.02, MeOH). ¹H NMR (501 MHz, CHLOROFORM-d) δ ppm 1.32 (s, 9H) 1.40-1.54 (m, 4H) 1.62-1.85 (m, 4H) 3.06-3.36 (m, 2H) 3.51 (t, J=6.78 Hz, 2H) 7.35-7.53 (m, 3H) 7.74-7.92 (m, 2H). ¹³C NMR (125 MHz, CHLOROFORM-d) δ ppm 179.8, 137.9, 131.4, 128.5, 127.4, 57.5, 44.9, 32.4, 29.0, 28.4, 26.5, 22.7. HRMS (EI) Calculated for C₁₇H₂₇ClNOS [M+H]: 328.1502. Found 328.1504

### (S,E)-2-methyl-N-(1-phenylbutylidene)propane-2-sulfinamide (1p)

Following the general procedure (GP1), the reaction of 1-phenylpentan-1-one (4p) (10 g, 67.47 mmol) with (S_{S})-tert-butanesulfinamide (12.16 g, 101.20 mmol) and Ti(OEt)₄ (30.786 g, 134.94 mmol) yields 15.6 g (92%) of pure γ-Chloro N-sulfinyl ketimine (S_{S}) 1p as a viscous liquid. [α]²⁵_{D}=+ 30.37°, (c 1.63, MeOH). ¹H NMR (501 MHz, CHLOROFORM-d) δ ppm 7.78-7.92 (m, 2H), 7.37-7.52 (m, 3H), 3.20-3.32 (m, 1H), 3.07-3.20 (m, 1H), 1.65-1.78 (m, 2H), 1.32 (s, 9H), 1.03 (t, J=7.41 Hz, 3H). ¹³C NMR (125 MHz, CHLOROFORM-d) δ ppm 180.0, 138.2, 131.4, 128.5, 127.4, 57.3, 34.3, 22.6, 22.2, 14.2. HRMS (EI) Calculated for C₁₄H₂₂NOS [M+H]: 252.1422, Found 252.1414.

### Example 2

### Asymmetric Reductive Cyclization of γ-Chlorinated N-Tert-Butanesulfinyl Ketimines (S_{S})-1a-1

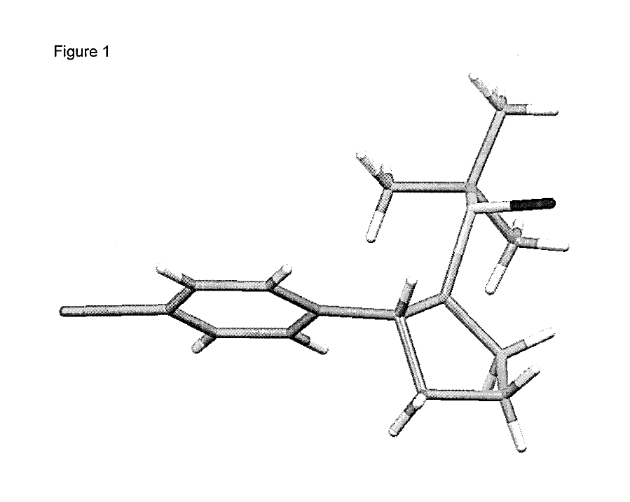

With the sulfinyl ketimines of Example 1, the preparation of 2-substituted pyrrolidines via asymmetric reduction of the γ-chloro N-tert-butanesulfinyl ketimines is explored. Reduction of sulfinyl ketimine (S_{S})-1 with 1.1 equiv of LiBHEt₃ in dry THF at −78° C. for 3 hour followed by extractive workup affords the crude γ-chloro sulfinamide (S_{S}, R)-6a in quantitative yield with little or no formation of the ring closed product, i.e., pyrrolidine (S_{S}, R)-2a (Scheme 3). Further stirring the crude γ-chloro sulfinamide 6a with a strong base like LiHMDS in THF at room temperature for 1 hour affords the desired pyrrolidine (S_{S}, R)-2a in 90% overall yield from 1a with a high diastereomeric ratio of 99:1 (Scheme 3). The diastereoselectivity of the reaction is determined based on ¹HNMR analysis of the crude product. The (R)-1-((S)-tert-butylsulfinyl)-2-(4-bromophenyl)pyrrolidine (2a) is obtained as a colorless crystalline solid (mp 102-104° C.). The structure and absolute configuration of (S_{S}, R)-2a was confirmed by single crystal X-ray diffraction analysis (FIG. 1). Further optimization of the two-step process to one-step is achieved by warming the reaction mixture at −78 to 23° C. followed by stirring for 1 hour, yielded pyrrolidine (S_{S}, R)-2a in a single step in 96% yield with a high diastereoselectivity of 99:1.

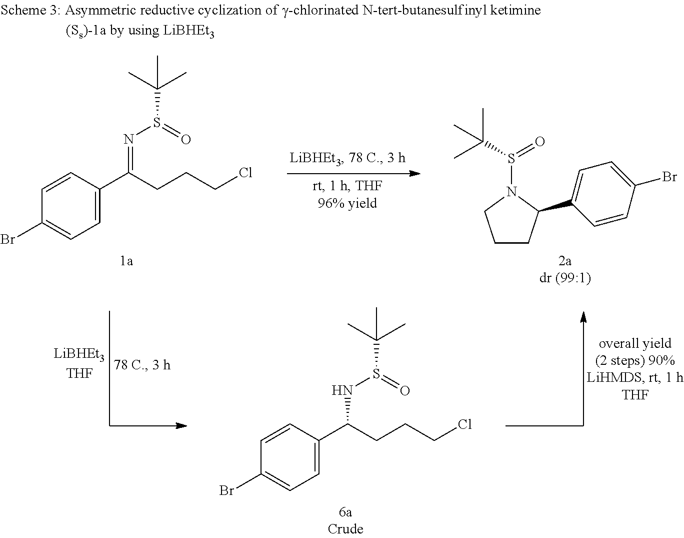

Based on these results, a series of metal hydrides are screened to further elaborate this reduction and test the possibility of stereoselectivity reversal. A series of reducing agents, such as L-Selectride, NaBH₄, LiBH₄, 9-BBN, NaBH₃CN, LiAlH₄, and DIBAL-H, are studied in the reduction of γ-chloro N-tert-butanesulfinyl ketimines 1b in THF for 3 hours at −78° C. Selectivity is monitored after cyclization of crude sulfinamide 6b to the desired crude pyrrolidines 2b and 3b by using 1.5 equivalents of LiHMDS. (Table 1). Reduction with L-Selectride provided a similar result as LiBHEt₃ (99:1 dr, Table 1, entry 1). Reversal of diastereoselectivity with a ratio of 40:60 (2b/3b) occurred when NaBH₄ was used as a reducing agent (Table 1, entry 2). Replacing NaBH₄ with LiBH₄ resulted in a slightly better outcome with a 35:65 dr (Table 1, entry 3). Reduction using a sterically hindered reducing agent like 9-borabicyclo[3.3.1]nonane (9-BBN) failed to show any improvement (38:62 dr, Table 1, entry 4). Interestingly, LiAlH₄ showed an improved diastereoselectivity of 18:82 dr, (Table 1, entry 6). When DIBAL-H was used, a marked enhancement in diastereoselectivity was observed with a diastereomeric ratio of 4:96 (Table 1, entry 7). The diastereoselectivity was further improved to 1:99 when the reduction was conducted in toluene instead of THF. (Table 1, entry 7).

TABLE 1
Reductive Cyclization of Sulfinyl Ketimine (Sₛ)-1b Using Various
Metal Hydrides

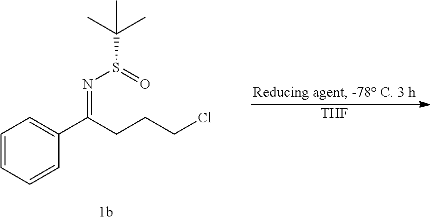

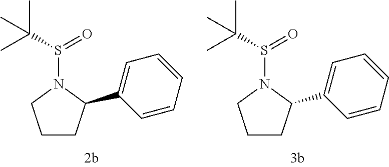

| Entry | Reducing agent | Solvent | Conversion (%) | 2b:3b^{a} |
|---|---|---|---|---|
| 1 | L-Selectride | THF | 100 | 99:1 |
| 2 | NaBH₄ | THF | 98 | 40:60 |
| 3 | LiBH₄ | THF | 100 | 35:65 |
| 4 | 9-BBN | THF | 100 | 38:62 |
| 5 | LiAlH₄ | THF | 100 | 18:82 |
| 6 | DIBAL-H | THF | 100 | 4:96 |
| 7 | DIBAL-H | Toluene | 100 | 1:99 |

^{a}All reactions were performed using 1.5 equiv of reducing agent at −78° C. for 3 hours, followed by isolation of crude product and cyclization using LiHMDS, room termperature, 1 hour in dry THF, unless stated otherwise indicated.

After obtaining these results, efforts are focused on developing a single step method to yield 3b with high diastereoselectivity. Unlike the LiBHEt₃ conditions, pyrrolidine 3b is not observed during the reduction of sulfinyl ketimine 1a with DIBAL-H at −78° C. for 3 hours followed by warming to room temperature and stirred for 1-12 hours. The addition of LiHMDS to the reaction mixture followed by warming the reaction mixture from about −78 to 23° C. led to complete cyclization. Thus, in a one pot, (S_{S},S)-3a in a 90% yield is synthesized with a high diastereomeric ratio of 1:99 (Scheme 4). The structure and absolute configuration of (S_{S},S)-3a was confirmed by comparing the ¹H NMR, ¹³C NMR and specific rotation data with literature data. (See, e.g., Reddy et al., Chemical Communications, 46(2), 222-224 (2010), which is incorporated herein by reference in its entirety.)

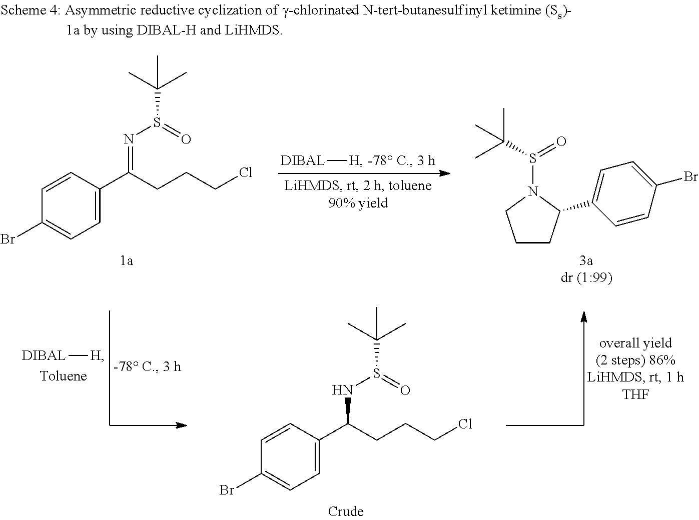

### Example 3

### Scope of the Reaction

With on optimized one step processes to access either of the two diastereomers of 2-substituted pyrrolidines (S_{S}, R)-2 and (S_{S},S)-3 starting from N-sulfinyl ketimines 1, a variety of γ-chlorinated N-tert-butanesulfinyl ketimines (S_{S})-1 are used as substrates to probe the general applicability (Table 2). In the LiBHEt₃ mediated reductive cyclization of phenyl γ-chloro N-tert-butanesulfinyl ketimine 1b, substituents such as OMethyl, Methyl (Me), t-Bu, OH, Cl, Br, F, etc. in the para and meta positions of the phenyl ring (1c-1i) are well tolerated and high diastereoselectivity (99:1 dr) and with high yields are obtained in every instance. (Table 2, entries 3-6). Similarly, treatment 1c-1i with DIBAL-H/LiHMDS affords the corresponding 2-aryl substituted pyrrolidines (3c-3i) in 88-94% yields with high diastereoselectivity (99:1 dr). Furthermore, heteroaryl ketimine 1j, when treated with LiBHEt₃ or DIBAL-H/LiHMDS, also undergo stereoselective reductive cyclization to afford 2j or 2j with diastereomeric ratio of 99:1 and 1:99 in 93 and 95% yields, respectively (Table 2, entry 10). In addition, these reactions are also found to be tolerant to aliphatic ketimines (Table 2, entries 11, 12). Treatment of cyclohexyl sulfinyl ketimine 1k with LiBHEt₃ or DIBAL-H/LiHMDS also affords the corresponding pyrrolidines 2k or 3k in 94 and 95% yield with diastereomeric ratio of 99:1 and 1:99, respectively (Table 2, entry 10). However, when methyl ketimine 1l is subjected to LiBHEt₃ conditions, the reverse stereoselective product, i.e., (R)-1-((S)-tert-butylsulfinyl)-2-methylpyrrolidine (S_{S},R-3l) is obtained with slightly lower diastereoselectivities (8:92 dr) in 88% yield (Table 2, entry 12). In the same way, treatment of 1l with DIBAL-H/LiHMDS affords the reverse stereoselective product, i.e., (S)-1-((S)-tert-butylsulfinyl)-2-methyl-pyrrolidine (S_{S},S-2l) with 89% yield and diastereomeric ratio of 91:9 (Table 2, entry 12). Thus, as summarized in Table 2, all the reactions take place readily and afford the corresponding pyrrolidines in good to excellent yields with high diastereoselectivities.

TABLE 2
Asymmetric reductive cyclization of γ-chlorinated N-tert-butanesulfinyl ketimine
(Sₛ)-1a-1l with LiBHEt₃ and DIBAL-H/LiHMDS

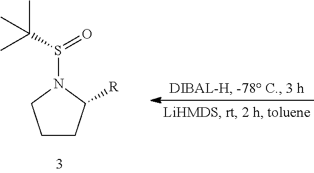

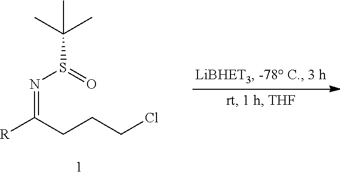

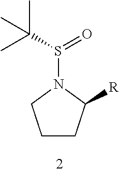

| | | LiBHEt₃ | | DIBAL-H | |
|---|---|---|---|---|---|
| Entry | R | Product | Yield^{a} (dr)^{b} | Product | Yield^{a} (dr)^{b} |
| 1 | 4-BrC₆H₄ | 2a | 96 (99:1) | 3a | 90 (1:99) |
| 2 | C₆H₅ | 2b | 94 (99:1) | 3b | 92 (1:99) |
| 3 | 4-MeC₆H₄ | 2c | 92 (99:1) | 3c | 93 (1:99) |
| 4 | 4-MeOC₆H₄ | 2d | 90 (99:1) | 3d | 87 (1:99) |
| 5 | 4-tBuC₆H₄ | 2e | 93 (99:1) | 3e | 91 (1:99) |
| 6 | 4-HOC₆H₄ | 2f | 98 (99:1) | 3f | 94 (1:99) |
| 7 | 3-MeOC₆H₄ | 2g | 92 (99:1) | 3g | 90 (1:99) |
| 8 | 4-ClC₆H₄ | 2h | 98 (99:1) | 3h | 93 (1:99) |
| 9 | 4-FC₆H₄ | 2i | 93 (99:1) | 3i | 98 (1:99) |
| 10 | 2-thienyl | 2j | 93 (99:1) | 3j | 95 (1:99) |
| 11 | C₆H₁₁ | 2k | 94 (99:1) | 3k | 95 (1:99) |
| 12 | Me | 2l | 88 (8:92) | 3l | 89 (91:9) |

^{a}Isolated yield of analytically pure products.
^{b}The diastereoselectivity was determined by ¹H NMR analysis.
The “99:1 or 1:99” denotes that signals for only one diastereomer were observed; dr relative to sulfur.

General Procedure (GP2) for the Synthesis 2-substituted pyrrolidines 2:

LiBHEt₃ is added to a solution of ketimine 1a (5 mmol) in THF (15 mL) at −78° C. under nitrogen. After stirring for 3 hours at −78° C., the reaction mixture is warmed up to room temperature and stirred for 1 hours. On completion, the reaction is quenched with saturated NH₄Cl solution (20 mL). The organic layer is then separated, washed with water and dried under vacuum to give crude product. The crude product is purified by column chromatography (silica gel, ethyl acetate/hexanes) to afford the pure 2-substituted pyrrolidines 2.

General Procedure (GP3) for the Synthesis of 2-Substituted Pyrrolidines 3:

To a solution of ketimine 1a (5 mmol) in Toluene (15 mL) at −78° C. DIBAL-His added under nitrogen. After stirring for 3 hours at −78° C., the reaction mixture was warmed up to room temperature followed by addition of LiHMDS (7.5 mmol) and stirred for 1 hour. On completion, the reaction is quenched with saturated K⁺Na⁺ tartarate solution (20 mL). The organic layer is then separated, washed with water and dried under vacuum to give crude product. The crude product is purified by column chromatography (silica gel, ethyl acetate/hexanes) to afford the pure 2-substituted pyrrolidines 3.

### (R)-1-((S)-2-Methyl-propane-2-sulfinyl)-2-(4-bromophenyl)-pyrrolidine (2a)

Following the general procedure (GP2), the reaction of γ-Chloro N-sulfinyl ketimine (S_{S}) as (1.82 g, 5.0 mmol) with LiBHEt₃ (6.0 mL, 6.0 mmol, 1.0 M in THF) affords pyrrolidine 2a (1.58 g, 96%) as white solid, mp=120-122° C., [α]²⁵_{D}=+ 152.3° (c 1.13, CHCl₃). ¹H NMR (501 MHz, CHLOROFORM-d) δ ppm 1.10 (s, 9H) 1.68-2.02 (m, 3H) 2.17-2.30 (m, 1H) 2.89-3.05 (m, 1H) 3.83-3.96 (m, 1H) 4.59 (t, J=7.25 Hz, 1H) 7.17 (d, J=8.51 Hz, 2H) 7.44 (d, J=8.51 Hz, 2H). ¹³C NMR (125 MHz, CHLOROFORM-d) δ ppm 142.3, 131.4, 128.9, 121.0, 68.7, 57.2, 42.1, 35.9, 26.3, 23.8. HRMS (EI) Calculated for C₁₄H₂₁BrNOS [M+H]: 330.0522. Found 330.0527.

### (S)-1-((S)-2-Methyl-propane-2-sulfinyl)-2-(4-bromophenyl)-pyrrolidine (3a)

Following the general procedure (GP3), the reaction of γ-Chloro N-sulfinyl ketimine (S_{S}) 1a (3.65 g, 10.0 mmol) with DIBAL-H (12.0 mL, 12.0 mmol, 1.0 M in Toluene) and followed by LiHMDS (15.0 ml, 15.5 mmol, 1.0 mL in THF) affords pyrrolidine 3a (2.91 g, 90%) as white solid, mp=100-112° C., [α]²⁵_{D}=−164.3° (c 1.13, CHCl₃). ¹H NMR (501 MHz, CHLOROFORM-d) δ ppm 1.05 (s, 9 H) 1.64-1.96 (m, 3H) 2.16 (dd, J=11.98, 9.14 Hz, 1H) 3.49-3.69 (m, 2H) 5.02 (dd, J=8.20, 2.84 Hz, 1H) 7.14 (d, J=8.51 Hz, 2H) 7.38-7.49 (m, 2H). ¹³C NMR (125 MHz, CHLOROFORM-d) δ ppm 143.7, 131.4, 128.2, 120.3, 57.4, 56.9, 54.8, 36.4, 24.1, 23.0. HRMS (EI) Calculated for C₁₄H₂₁BrNOS [M+H]: 330.0527. Found 330.0539.

### (R)-1-((S)-2-Methyl-propane-2-sulfinyl)-2-phenyl-pyrrolidine (2b)

Following the general procedure (GP2), the reaction of γ-Chloro N-sulfinyl ketimine (S_{S}) 1b (1.42 g, 5.0 mmol) with LiBHEt₃ (6.0 mL, 6.0 mmol, 1.0 M in THF) affords pyrrolidine 2b (1.17 g, 94%) as a viscous liquid. [α]²⁵_{D}=+121.6° (c 1.05, CHCl₃). ¹H NMR (501 MHz, CHLOROFORM-d) δ ppm 1.10 (s, 9H) 1.71-2.01 (m, 3H) 2.19-2.34 (m, 1H) 2.91-3.04 (m, 1H) 3.83-3.99 (m, 1H) 4.64 (t, J=7.25 Hz, 1H) 7.14-7.40 (m, 5H). ¹³C NMR (125 MHz, CHLOROFORM-d) δ ppm 143.3, 128.2, 127.2, 69.3, 57.2, 42.1, 35.9, 26.3, 23.8. HRMS (EI) Calculated for C₁₄H₂₂NOS [M+H]: 252.1439. Found 252.1422.

### (S)-1-((S)-2-Methyl-propane-2-sulfinyl)-2-phenyl-pyrrolidine (3b)

Following the general procedure (GP3), the reaction of γ-Chloro N-sulfinyl ketimine (S_{S}) 1b (1.42 g, 5.0 mmol) with DIBAL-H (6.0 mL, 6.0 mmol, 1.0 M in Toluene) and followed by LiHMDS (7.5 ml, 7.5 mmol, 1.0 mL in THF) affords pyrrolidine 3a (1.15 g, 92%) as white solid, mp=80-81° C., [α]²⁵_{D}=−140.4° (c 1.07, CHCl₃). ¹H NMR (501 MHz, CHLOROFORM-d) δ ppm 1.05 (s, 9H) 1.71-1.92 (m, 3H) 2.16 (dd, J=11.35, 8.51 Hz, 1H) 3.52-3.60 (m, 1H) 3.61-3.71 (m, 1H) 5.07 (dd, J=8.04, 2.68 Hz, 1H) 7.16-7.34 (m, 5H). ¹³C NMR (125 MHz, CHLOROFORM-d) δ ppm 143.1, 126.8, 125.0, 55.9, 55.9, 53.3, 35.1, 22.6, 21.5. HRMS (EI) Calculated for C₁₄H₂₂NOS [M+H]: 252.1402. Found 252.1413.

### (R)-1-((S)-2-Methyl-propane-2-sulfinyl)-2-(4-methylphenyl)-pyrrolidine (2c)

Following the general procedure (GP2), the reaction of γ-Chloro N-sulfinyl ketimine (S_{S}) 1c (1.49 g, 5.0 mmol) with LiBHEt₃ (6.0 mL, 6.0 mmol, 1.0 M in THF) affords pyrrolidine 2c (1.21 g, 92%) as a white solid. mp=62-64° C., [α]²⁵_{D}=+ 144.66° (c 1.03, MeOH). ¹H NMR (501 MHz, CHLOROFORM-d) δ ppm 7.17 (d, J=8.2 Hz, 2H), 7.11 (d, J=8.2 Hz, 2H), 4.60 (t, J=7.41 Hz, 1H), 3.83-3.92 (m, 1H), 2.92-3.01 (m, 1H), 2.32 (s, 3H), 2.15-2.25 (m, 1H), 1.93-2.01 (m, 1H), 1.72-1.90 (m, 2H), 1.10 (s, 9H). ¹³C NMR (125 MHz, CHLOROFORM-d) δ ppm 140.2, 136.7, 128.9, 127.1, 69.0, 57.1, 42.0, 35.9, 26.3, 23.8, 21.0. HRMS (EI) Calculated for C₁₅H₂₄NOS [M+H]: 266.1538. Found 266.1581.

### (S)-1-((S)-2-Methyl-propane-2-sulfinyl)-2-(4-methylphenyl)-pyrrolidine (3c)

Following the general procedure (GP3), the reaction of γ-Chloro N-sulfinyl ketimine (S_{S}) 1c (1.49 g, 5.0 mmol) with DIBAL-H (6.0 mL, 6.0 mmol, 1.0 M in Toluene) and followed by LiHMDS (7.5 ml, 7.5 mmol, 1.0 mL in THF) affords pyrrolidine 3c (1.39 g, 93%) as white solid, mp=96-98° C., [α]²⁰_{D}=−159° (c 0.8, MeOH). ¹H NMR (501 MHz, CHLOROFORM-d) δ ppm 7.09-7.17 (m, 4H), 5.02 (dd, J=8.04, 2.68 Hz, 1H), 3.62-3.70 (m, 1H), 3.50-3.57 (m, 1H), 2.32 (s, 3H), 2.08-2.18 (m, 1H), 1.77-1.90 (m, 2H), 1.70-1.78 (m, 1H), 1.06 (s, 9H). ¹³C NMR (125 MHz, CHLOROFORM-d) δ ppm 141.5, 136.0, 129.0, 126.4, 57.5, 57.4, 54.5, 36.6, 24.1, 23.1, 23.0. HRMS (EI) Calculated for C₁₅H₂₄NOS [M+H]: 266.1579. Found 266.1569.

### (R)-1-((S)-2-Methyl-propane-2-sulfinyl)-2-(4-methoxyphenyl)-pyrrolidine (2d)

Following the general procedure (GP2), the reaction of γ-Chloro N-sulfinyl ketimine (S_{S}) 1d (1.57 g, 5.0 mmol) with LiBHEt₃ (6.0 mL, 6.0 mmol, 1.0 M in THF) afforded pyrrolidine 2d (1.26 g, 90%) as a white solid. mp=61-63° C., [α]²⁵_{D}=+123.2° (c 1.0, MeOH). ¹H NMR (400 MHz, CHLOROFORM-d) ppm 7.18-7.23 (m, 2H), 6.82-6.87 (m, 2H), 4.52-4.61 (m, 1H), 3.83-3.90 (m, 1H), 3.78 (s, 3H), 2.91-3.00 (m, 1H), 2.14-2.24 (m, 1H), 1.92-2.02 (m, 1H), 1.70-1.90 (m, 2H), 1.09 (s, 9H). ¹³C NMR (125 MHz, CHLOROFORM-d) δ ppm 158.7, 135.0, 128.3, 113.6, 68.6, 57.0, 55.1, 41.9, 35.9, 26.3, 23.8. HRMS (EI) Calculated for C₁₅H₂₄NO₂S [M+H]: 282.1528. Found 282.1527.

### (S)-1-((S)-2-Methyl-propane-2-sulfinyl)-2-(4-methoxyphenyl)-pyrrolidine (3d)

Following the general procedure (GP3), the reaction of γ-Chloro N-sulfinyl ketimine (S_{S}) 1d (1.57 g, 5.0 mmol) with DIBAL-H (6.0 mL, 6.0 mmol, 1.0 M in Toluene) and followed by LiHMDS (7.5 ml, 7.5 mmol, 1.0 mL in THF) affords pyrrolidine 3d (1.22 g, 87%) as white solid, mp=85-87° C., [α]²⁵_{D}=−140.82° (c 1.09, MeOH). ¹H NMR (501 MHz, CHLOROFORM-d) δ ppm 7.16 (d, J=8.83 Hz, 2 H), 6.83-6.87 (m, 2H), 4.99 (dd, J=7.88, 2.84 Hz, 1H), 3.79 (s, 3H), 3.62-3.70 (m, 1H), 3.47-3.55 (m, 1H), 2.07-2.17 (m, 1H), 1.78-1.91 (m, 2H), 1.69-1.76 (m, 1H), 1.06 (s, 9H). ¹³C NMR (125 MHz, CHLOROFORM-d) δ ppm 158.2, 136.6, 127.6, 113.7, 57.4, 55.1, 54.2, 36.6, 24.1, 23.1. HRMS (EI) Calculated for C₁₅H₂₄NO₂S [M+H]: 282.1528. Found 282.1559.

### (R)-1-((S)-2-Methyl-propane-2-sulfinyl)-2-(4-tert-butylphenyl)-pyrrolidine (2e)

Following the general procedure (GP2), the reaction of γ-Chloro N-sulfinyl ketimine (S_{S}) 1e (1.70 g, 5.0 mmol) with LiBHEt₃ (6.0 mL, 6.0 mmol, 1.0 M in THF) affords pyrrolidine 2e (1.42 g, 93%) as a white solid. mp=45-50° C. [α]²⁵_{D}=110.5° (c 1.0, MeOH). ¹H NMR (400 MHz, CHLOROFORM-d) S ppm 7.33 (d, J=8.34 Hz, 2H), 7.21 (d, J=8.34 Hz, 2H), 4.64 (t, J=6.95 Hz, 1H), 3.85-3.92 (m, 1H), 2.93-3.01 (m, 1H), 2.17-2.25 (m, 1H), 1.91-2.00 (m, 1H), 1.74-1.91 (m, 2H), 1.31 (s, 9H), 1.11 (s, 9H). ¹³C NMR (125 MHz, CHLOROFORM-d) δ ppm 150.0, 140.1, 126.7, 125.1, 57.2, 42.0, 35.8, 34.4, 31.5, 31.4, 26.2, 23.9. HRMS (EI) Calculated for C₁₈H₃₀NOS [M+H]:308.2048. Found 308.2011.

### (S)-1-((S)-2-Methyl-propane-2-sulfinyl)-2-(4-tert-butylphenyl)-pyrrolidine (3e)

Following the general procedure (GP3), the reaction of γ-Chloro N-sulfinyl ketimine (S_{S}) 1e (1.70 g, 5.0 mmol) with DIBAL-H (6.0 mL, 6.0 mmol, 1.0 M in Toluene) and followed by LiHMDS (7.5 ml, 7.5 mmol, 1.0 mL in THF) affords pyrrolidine 3e (1.39 g, 91%) as white solid, mp=55-60° C., [α]²⁵_{D}=−141.07° (c 1.12, MeOH). ¹H NMR (501 MHz, CHLOROFORM-d) δ ppm 7.31 (d, J=8.20 Hz, 2 H), 7.16 (d, J=8.20 Hz, 2H), 5.02 (dd, J=8.04, 2.36 Hz, 1H), 3.63-3.70 (m, 1H), 3.48-3.54 (m, 1H), 2.09-2.16 (m, 1H), 1.74-1.88 (m, 3H), 1.31 (s, 9H), 1.07 (s, 9H). ¹³C NMR (125 MHz, CHLOROFORM-d) δ ppm 149.3, 141.2, 126.1, 125.1, 57.8, 57.4, 54.1, 36.4, 34.4, 31.3, 24.1, 23.1. HRMS (EI) Calculated for C₁₈H₃₀NOS [M+H]: 308.2048. Found 308.2043.

### (R)-1-((S)-2-Methyl-propane-2-sulfinyl)-2-(4-hydroxylphenyl)-pyrrolidine (2f)

Following the general procedure (GP2), the reaction of γ-Chloro N-sulfinyl ketimine (S_{S}) 1f (1.50 g, 5.0 mmol) with LiBHEt₃ (6.0 mL, 6.0 mmol, 1.0 M in THF) affords pyrrolidine 2f (1.30 g, 98%) as a white solid, mp=150° C., [α]²⁵_{D}=+137.4° (c 0.93, MeOH). ¹H NMR (501 MHz, CHLOROFORM-d) δ ppm 7.13 (d, J=8.51 Hz, 2H), 6.82 (d, J=8.51 Hz, 2H), 6.50 (br. s., 1H), 4.53-4.56 (m, 1H), 3.84-3.89 (m, 1H), 2.94-3.01 (m, 1H), 2.18-2.23 (m, 1H), 1.95-2.01 (m, 1H), 1.74-1.87 (m, 2H), 1.12 (s, 9H). ¹³C NMR (125 MHz, CHLOROFORM-d) δ ppm 155.6, 134.4, 128.5, 115.2, 68.7, 57.2, 42.2, 35.7, 26.3, 23.8. HRMS (EI) Calculated for C₁₄H₂₂NO₂S [M+H]: 268.1371. Found 268.1364.

### (S)-1-((S)-2-Methyl-propane-2-sulfinyl)-2-(4-hydroxyphenyl)-pyrrolidine (3f)

Following the general procedure (GP3), the reaction of γ-Chloro N-sulfinyl ketimine (S_{S}) 1f (1.50 g, 5.0 mmol) with DIBAL-H (6.0 mL, 6.0 mmol, 1.0 M in Toluene) and followed by LiHMDS (7.5 ml, 7.5 mmol, 1.0 mL in THF) affords pyrrolidine 3f (1.25 g, 94%) as white solid, mp=185° C., [α]²⁵_{D}=−124.4° (c 1.02, MeOH). ¹H NMR (501 MHz, CHLOROFORM-d) δ ppm 8.07 (s, 1H), 7.08 (d, J=8.51 Hz, 2H), 6.83 (d, J=8.83 Hz, 2H), 4.87 (dd, J=7.57, 3.15 Hz, 1H), 3.69-3.76 (m, 1H), 3.40-3.47 (m, 1H), 2.13 (dd, J=11.98, 7.88 Hz, 1H), 1.81-1.92 (m, 2H), 1.73-1.81 (m, 1H), 1.13 (s, 9H). ¹³C NMR (125 MHz, CHLOROFORM-d) δ ppm 155.8, 134.6, 127.8, 115.5, 60.0, 57.8, 51.9, 36.2, 24.0, 23.4. HRMS (EI) Calculated for C₁₄H₂₂NO₂S [M+H]: 268.1371. Found 268.1365.

### (R)-1-((S)-2-Methyl-propane-2-sulfinyl)-2-(3-methoxyphenyl)-pyrrolidine (2g)

Following the general procedure (GP2), the reaction of γ-Chloro N-sulfinyl ketimine (S_{S}) 1g (1.57 g, 5.0 mmol) with LiBHEt₃ (6.0 mL, 6.0 mmol, 1.0 M in THF) affords pyrrolidine 2g (1.28 g, 92%) as a white solid, mp=45-50° C., [α]²⁵_{D}=+152.8° (c 1.12, MeOH). ¹H NMR (501 MHz, CHLOROFORM-d) δ ppm 7.23 (t, J=7.88 Hz, 1H), 6.82-6.91 (m, 2H), 6.75-6.81 (m, 1H), 4.63 (t, J=7.09 Hz, 1H), 3.86-3.92 (m, 1H), 3.80 (s, 3H), 2.95-3.00 (m, 1H), 2.22-2.27 (m, 1H), 1.94-2.00 (m, 1H), 1.77-1.89 (m, 2H), 1.12 (s, 9H). ¹³C NMR (125 MHz, CHLOROFORM-d) δ ppm 159.2, 145.0, 129.3, 119.5, 112.9, 112.4, 69.2, 57.2, 55.1, 42.1, 35.8, 26.2, 23.8. HRMS (EI) Calculated for C₁₅H₂₄NO₂S [M+H]: 282.1528. Found 282.1519.

### (S)-1-((S)-2-Methyl-propane-2-sulfinyl)-2-(3-methoxyphenyl)-pyrrolidine (3g)

Following the general procedure (GP3), the reaction of γ-Chloro N-sulfinyl ketimine (S_{S}) 1g (1.57 g, 5.0 mmol) with DIBAL-H (6.0 mL, 6.0 mmol, 1.0 M in Toluene) and followed by LiHMDS (7.5 ml, 7.5 mmol, 1.0 mL in THF) affords pyrrolidine 3g (1.26 g, 90%) as a viscous liquid, [α]²⁵_{D}=−131.04° (c 1.21, MeOH). ¹H NMR (501 MHz, CHLOROFORM-d) δ ppm 7.22 (t, J=7.88 Hz, 1H), 6.80-6.86 (m, 2H), 6.75 (dd, J=8.20, 2.52 Hz, 1H), 5.03-5.05 (m, 1H), 3.80 (s, 3H), 3.61-3.68 (m, 1 H), 3.53-3.58 (m, 1H), 2.11-2.19 (m, 1H), 1.72-1.92 (m, 3H), 1.07 (s, 9H). ¹³C NMR (125 MHz, CHLOROFORM-d) δ ppm 159.6, 146.4, 129.4, 118.9, 112.4, 111.6, 57.4, 55.1, 54.9, 36.5, 24.2, 23.0. HRMS (EI) Calculated for C₁₅H₂₄NO₂S [M+H]: 282.1528. Found 282.1516.

### (R)-1-((S)-2-Methyl-propane-2-sulfinyl)-2-(4-chlorophenyl)-pyrrolidine (2h)

Following the general procedure (GP2), the reaction of γ-Chloro N-sulfinyl ketimine (S_{S}) 1h (1.59 g, 5.0 mmol) with LiBHEt₃ (6.0 mL, 6.0 mmol, 1.0 M in THF) affords pyrrolidine 2h (1.39 g, 98%) as a white solid, mp=75-77° C., [α]²⁵_{D}=+111.98° (c 1.09, MeOH). ¹H NMR (400 MHz, CHLOROFORM-d) δ ppm 7.21 (dd, 4H), 4.55 (t, 1H), 3.79-3.87 (m, 1H), 2.87-2.96 (m, 1H), 2.14-2.23 (m, 1 H), 1.63-1.97 (m, 3H), 1.05 (s, 9H). ¹³C NMR (125 MHz, CHLOROFORM-d) δ ppm 141.8, 132.9, 128.6, 128.4, 68.6, 57.2, 42.1, 36.0, 26.3, 23.8. HRMS (EI) Calculated for C₁₄H₂₁NOSCl [M+H]: 286.1032. Found 286.1028.

### (S)-1-((S)-2-Methyl-propane-2-sulfinyl)-2-(4-chlorophenyl)-pyrrolidine (3h)

Following the general procedure (GP3), the reaction of γ-Chloro N-sulfinyl ketimine (S_{S}) 1h (1.59 g, 5.0 mmol) with DIBAL-H (6.0 mL, 6.0 mmol, 1.0 M in Toluene) and followed by LiHMDS (7.5 ml, 7.5 mmol, 1.0 mL in THF) affords pyrrolidine 3h (1.32 g, 93%) as white solid, mp=95-102° C., [α]²⁵_{D}=−145.6° (c 0.75, MeOH). ¹H NMR (400 MHz, CHLOROFORM-d) δ ppm 7.26-7.31 (m, 2H), 7.16-7.21 (m, 2H), 5.04 (dd, J=7.96, 2.65 Hz, 1H), 3.50-3.69 (m, 1H), 2.07-2.23 (m, 1H), 1.65-1.94 (m, 3H), 1.05 (s, 9H), 1.05 (s, 9H). ¹³C NMR (125 MHz, CHLOROFORM-d) δ ppm 143.2, 132.2, 128.5, 127.9, 57.5, 56.9, 54.9, 36.5, 24.1, 23.0. HRMS (EI) Calculated for C₁₄H₂₁NOSCl [M+H]: 286.1032. Found 286.1034.

### (R)-1-((S)-2-Methyl-propane-2-sulfinyl)-2-(4-fluorophenyl)-pyrrolidine (2i)

Following the general procedure (GP2), the reaction of γ-Chloro N-sulfinyl ketimine (S_{S}) 1i (1.51 g, 5.0 mmol) with LiBHEt₃ (6.0 mL, 6.0 mmol, 1.0 M in THF) affords pyrrolidine 2i (1.25 g, 93%) as a white solid, [α]²⁵_{D}=+127.4° (c 1.18, MeOH). ¹H NMR (400 MHz, CHLOROFORM-d) δ ppm 7.21-7.30 (m, 2H), 6.95-7.04 (m, 2H), 4.58-4.64 (m, 1H), 3.82-3.93 (m, 1H), 2.92-3.01 (m, 1 H), 2.20-2.29 (m, 1H), 1.93-2.01 (m, 1H), 1.70-1.91 (m, 2H), 1.09 (s, 9H). ¹³C NMR (125 MHz, CHLOROFORM-d) δ ppm 163.2, 160.8, 138.9, 128.8, 128.7, 115.2, 115.0, 68.5, 57.2, 42.0, 36.0, 26.3, 23.8. HRMS (EI) Calculated for C₁₄H₂₁NOSF [M+H]: 270.1328. Found 270.1289.

### (S)-1-((S)-2-Methyl-propane-2-sulfinyl)-2-(4-chlorophenyl)-pyrrolidine (3i)

Following the general procedure (GP3), the reaction of γ-Chloro N-sulfinyl ketimine (S_{S}) 1i (1.51 g, 5.0 mmol) with DIBAL-H (6.0 mL, 6.0 mmol, 1.0 M in Toluene) and followed by LiHMDS (7.5 ml, 7.5 mmol, 1.0 mL in THF) affords pyrrolidine 3i (1.31 g, 98%) as white solid, mp=80° C., [α]²⁵_{D}=−146.5° (c 1.0, MeOH). ¹H NMR (501 MHz, CHLOROFORM-d) δ ppm 7.19-7.24 (m, 2H), 6.97-7.02 (m, 2H), 5.04 (dd, J=7.88, 2.84 Hz, 1H), 3.62-3.69 (m, 1H), 3.51-3.56 (m, 1H), 2.12-2.19 (m, 1H), 1.76-1.92 (m, 2H), 1.68-1.76 (m, 1H), 1.05 (s, 9H). ¹³C NMR (125 MHz, CHLOROFORM-d) δ ppm 162.5, 160.5, 140.3, 128.0, 128.0, 115.2, 115.0, 57.4, 57.0, 54.5, 36.5, 24.1, 23.0. HRMS (EI) Calculated for C₁₄H₂₁NOSF [M+H]: 270.1328. Found 270.1289.

### (R)-1-((S)-2-Methyl-propane-2-sulfinyl)-2-thiophenyl-pyrrolidine (2j)

Following the general procedure (GP2), the reaction of γ-Chloro N-sulfinyl ketimine (S_{S}) 1j (1.45 g, 5.0 mmol) with LiBHEt₃ (6.0 mL, 6.0 mmol, 1.0 M in THF) affords pyrrolidine 2j (1.19 g, 93%) as a viscous liquid, [α]²⁵_{D}=+ 98.50° (c 1.21, MeOH). ¹H NMR (501 MHz, CHLOROFORM-d) δ ppm 7.19 (dd, J=4.26, 1.73 Hz, 1H), 6.90-6.96 (m, 2H), 4.94 (t, J=6.46 Hz, 1H), 3.78-3.86 (m, 1H), 2.91-2.98 (m, 1H), 2.22-2.30 (m, 1H), 1.85-2.03 (m, 3H), 1.14 (s, 9H). ¹³C NMR (125 MHz, CHLOROFORM-d) δ ppm 147.9, 126.6, 124.4, 65.1, 57.5, 41.4, 35.8, 26.7, 23.7. HRMS (EI) Calculated for C₁₂H₂₀NOS₂ [M+H]: 258.0986. Found 258.0979.

### (S)-1-((S)-2-Methyl-propane-2-sulfinyl)-2thiophenyl-pyrrolidine (3j)

Following the general procedure (GP3), the reaction of γ-Chloro N-sulfinyl ketimine (S_{S}) 1j (1.45 g, 5.0 mmol) with DIBAL-H (6.0 mL, 6.0 mmol, 1.0 M in Toluene) and followed by LiHMDS (7.5 ml, 7.5 mmol, 1.0 mL in THF) affords pyrrolidine 3j (1.22 g, 95%) as white solid, mp=125-130° C., [α]²⁵_{D}=−130.25° (c 0.98, MeOH). ¹H NMR (501 MHz, CHLOROFORM-d) δ ppm 7.15 (d, J=5.04 Hz, 1H), 6.91-6.95 (m, 1H), 6.87 (d, J=3.15 Hz, 1H), 5.24-5.28 (m, 1H), 3.60-3.66 (m, 1H), 3.43-3.49 (m, 1H), 2.07-2.15 (m, 1H), 1.87-1.96 (m, 3H), 1.12 (s, 9H). ¹³C NMR (125 MHz, CHLOROFORM-d) δ ppm 148.9, 126.7, 123.8, 123.6, 57.6, 54.1, 53.9, 36.7, 24.2, 22.9. HRMS (EI) Calculated for C₁₂H₂₀NOS₂ [M+H]: 258.0986. Found 258.0953.

### (R)-1-((S)-2-Methyl-propane-2-sulfinyl)-2-cyclohexyl-pyrrolidine (2k)

Following the general procedure (GP2), the reaction of γ-Chloro N-sulfinyl ketimine (S_{S}) 1k (1.44 g, 5.0 mmol) with LiBHEt₃ (6.0 mL, 6.0 mmol, 1.0 M in THF) afforded pyrrolidine 2k (1.20 g, 94%) as a viscous liquid, [α]²⁵_{D}=+ 106° (c 0.75, MeOH). 1H NMR (501 MHz, CHLOROFORM-d) δ ppm 0.89-1.05 (m, 2H) 1.07-1.18 (m, 3H) 1.21 (s, 9H) 1.40-1.51 (m, 1H) 1.54-1.63 (m, 1H) 1.65-1.72 (m, 4H) 1.73-1.83 (m, 4H) 2.59-2.69 (m, 1H) 3.45-3.52 (m, 1H) 3.68-3.79 (m, 1H). ¹³C NMR (125 MHz, CHLOROFORM-d) δ ppm 70.7, 57.7, 42.6, 42.3, 30.4, 27.2, 26.8, 26.7, 26.6, 26.5, 26.2, 24.0. HRMS (EI) Calculated for C₁₄H₂₈NOS [M+H]: 258.1892. Found 258.1890.

### (S)-1-((S)-2-Methyl-propane-2-sulfinyl)-2-cyclohexyl pyrrolidine (3k)

Following the general procedure (GP3), the reaction of γ-Chloro N-sulfinyl ketimine (S_{c}) 1k (1.45 g, 5.0 mmol) with DIBAL-H (6.0 mL, 6.0 mmol, 1.0 M in Toluene) and followed by LiHMDS (7.5 mL, 7.5 mmol, 1.0 M in THF) affords pyrrolidine 3k (1.22 g, 95%) as a viscous liquid, [α]²⁵_{D}=−40.9°. (c 1.05, CHCl₃). 1H NMR (501 MHz, CHLOROFORM-d) δ ppm 0.89-1.06 (m, 2H) 1.05-1.17 (m, 1H) 1.20 (s, 9H) 1.23-1.32 (m, 2H) 1.60-1.83 (m, 10H) 3.14-3.23 (m, 1H) 3.30-3.39 (m, 1H) 3.53-3.63 (m, 1H). ¹³C NMR (125 MHz, CHLOROFORM-d) δ 62.7, 57.5, 50.0, 41.5, 30.7, 27.4, 27.2, 26.6, 26.5, 26.3, 25.2, 23.4. HRMS (EI) Calculated for C₁₄H₂₈NOS [M+H]: 258.1892. Found 258.1858.

### (S)-1-((S)-2-Methyl-propane-2-sulfinyl)-2-methyl-pyrrolidine (2l)

Following the general procedure (GP2), the reaction of γ-Chloro N-sulfinyl ketimine (S_{S}) 1l (1.11 g, 5.0 mmol) with LiBHEt₃ (6.0 mL, 6.0 mmol, 1.0 M in THF) affords pyrrolidine 2l (0.83 g, 88%) as a viscous liquid. [α]²⁵_{D}=+ 32.54° (c 0.89, MeOH). 1H NMR (501 MHz, CHLOROFORM-d) δ ppm 1.19 (s, 9H) 1.22 (d, J=6.62 Hz, 3H) 1.47-1.54 (m, 1H) 1.77-1.95 (m, 3H) 3.04-3.14 (m, 1H) 3.49-3.57 (m, 1H) 3.77-3.84 (m, 1H). ¹³C NMR (125 MHz, CHLOROFORM-d) δ ppm 60.7, 57.2, 54.7, 47.1, 41.3, 33.5, 24.0, 23.5, 20.4. HRMS (EI) Calculated for C₉H₂₀NOS [M+H]: 190.1258. Found 190.1266.

### (R)-1-((S)-2-Methyl-propane-2-sulfinyl)-2-methyl-pyrrolidine (3l)

Following the general procedure (GP3), the reaction of γ-Chloro N-sulfinyl ketimine (S_{S}) 1l (1.11 g, 5.0 mmol) with DIBAL-H (6.0 mL, 6.0 mmol, 1.0 M in Toluene) and followed by LiHMDS (7.5 mL, 7.5 mmol, 1.0 M in THF) affords pyrrolidine 3l (0.85 g, 89%) as a viscous liquid. [α]²⁵_{D}=+83.68° (c 1.38, CHCl₃). 1H NMR (501 MHz, CHLOROFORM-d) δ ppm 1.17 (d, J=5.99 Hz, 3H) 1.18 (s, 9H) 1.34-1.45 (m, 1H) 1.67-1.78 (m, 1H) 1.82-1.91 (m, 1H) 1.96-2.04 (m, 1H) 2.73-2.80 (m, 1H) 3.69-3.75 (m, 1H). ¹³C NMR (125 MHz, CHLOROFORM-d) δ ppm 60.6, 56.6, 41.2, 33.7, 25.9, 23.7, 22.0. HRMS (EI) Calculated for C₉H₂₀NOS [M+H]: 190.1258. Found 190.1260.

### Example 4

### Asymmetric Synthesis of 2-Substituted Piperidines

This methodology is investigated for extension to asymmetric synthesis of 2-substituted piperidines. Reduction of δ-chlorinated N-tert-butanesulfinyl ketimine (1m) with LiBHEt₃ at −78° C. for 3 hours followed by increasing the reaction temperature to room temperature and stirring overnight led to the desired 2-phenyl piperidine 7m with high diastereoselectivity (99:1) and 94% yield (Scheme 5). Likewise, treatment of 1m with DIBAL-H/LiHMDS afforded reversal of diastereofacial selectivity, and 2-phenyl piperidine 8m was obtained with dr (1:99) in 92% yield (Scheme 5).

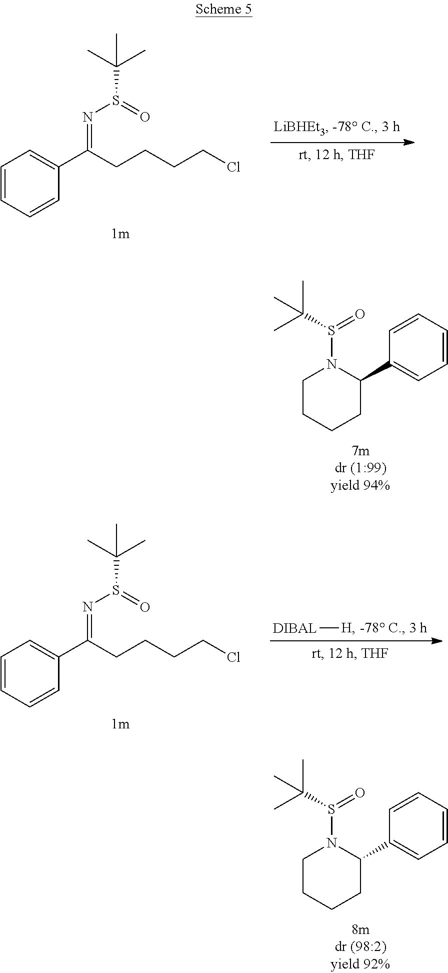

The basis for the reversal of diastereofacial selectivity upon changing reducing agents from LiBHEt₃ to DIBAL-H, is pursued. To exclude the role of the chloro group in the diastereoselectivity of the reducing agents, the dechlorinated ketimine 1p (Scheme 1) is synthesized. Reduction of 1p with DIBAL-H and LiBHEt₃ using the conditions mentioned earlier results in the formation of 9p and 10p respectively with high diastereoselectivity (Scheme 6), demonstrating that the chloro group does not play a role. Without being held to any theory, it is proposed that the DIBAL-H reaction proceeds through a closed transition state where the aluminium metal coordinates with the sulfinyl oxygen directing the hydride attack from the si face of the imine bond to give the (S_{S},S) diastereomer (FIG. 3, TTS 1). Alternatively, poorly coordinating and rapidly reacting LiBHEt₃ is posed to attack the electrophilic carbon atom in a sterically controlled fashion via an open transition state. Hence, delivery of the hydride would occur from the same face as the sulfur lone pair to give the (S_{S}, R) diastereomer (FIG. 3, TTS 2).

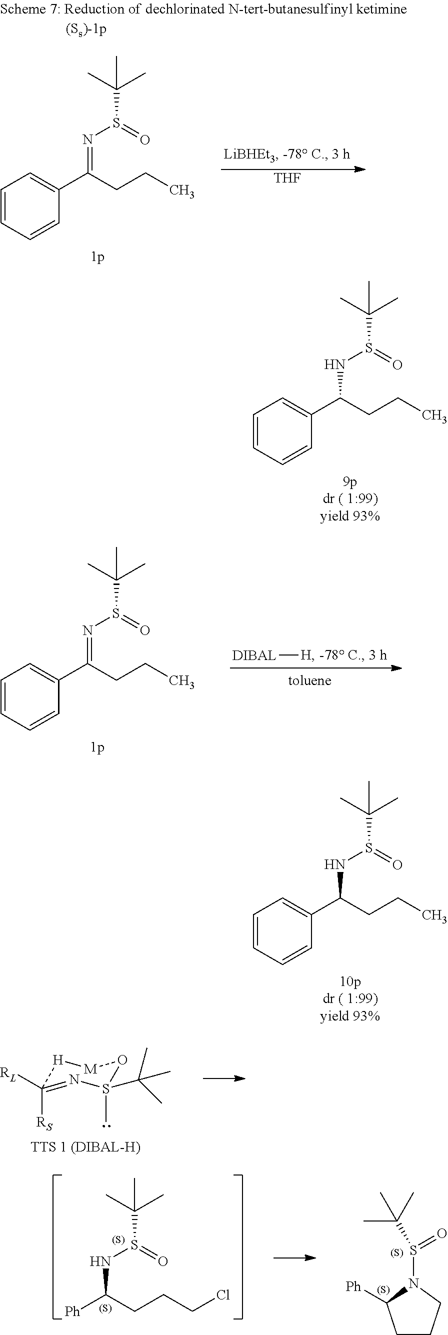

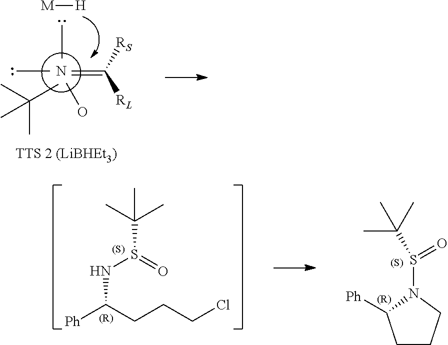

FIG. 3. Proposed transition states for the reduction of sulfinyl ketimines (S_{S})-1

### (R)-1-((S)-2-Methyl-propane-2-sulfinyl)-2-phenyl-piperidine (7m)

Following the general procedure (GP2), the reaction of γ-Chloro N-sulfinyl ketimine (S_{c}) 1m (1.49 g, 5.0 mmol) with LiBHEt₃ (6.0 mL, 6.0 mmol, 1.0 M in THF) affords piperidine 7m (1.24 g, 94%) as a white solid, mp=45-50° C., [α]²⁵_{D}=−116.96° (c 1.16, MeOH). ¹H NMR (501 MHz, CHLOROFORM-d) δ ppm 7.43 (d, J=8.20 Hz, 2H), 7.36 (t, J=7.72 Hz, 2H), 7.20-7.27 (m, 1H), 4.68 (t, J=4.10 Hz, 1H), 3.29 (dd, J=8.04, 3.31 Hz, 2H), 2.16-2.23 (m, 1H), 2.00-2.09 (m, 1H), 1.56-1.70 (m, 4 H), 1.42-1.52 (m, 1H), 1.15 (s, 9H). ¹³C NMR (125 MHz, CHLOROFORM-d) δ ppm 140.2, 128.5, 127.4, 126.6, 58.7, 31.2, 25.6, 23.0, 20.4. HRMS (EI) Calculated for C₁₅H₂₄NOS [M+H]: 266.1579. Found 266.1563.

### (S)-1-((S)-2-Methyl-propane-2-sulfinyl)-2-phenyl-piperidine (8m)

Following the general procedure (GP3), the reaction of γ-Chloro N-sulfinyl ketimine (S_{c}) 1m (1.49 g, 5.0 mmol) with DIBAL-H (6.0 mL, 6.0 mmol, 1.0 M in Toluene) and followed by LiHMDS (7.5 ml, 7.5 mmol, 1.0 mL in THF) affords piperidine 8m (1.21 g, 92%) as white solid, mp=50-55° C., [α]²⁵_{D}=108.07° (c 1.04, MeOH). ¹H NMR (501 MHz, CHLOROFORM-d) δ ppm 7.22-7.36 (m, 5H), 4.14 (dd, J=8.51, 4.10 Hz, 1H), 3.31-3.36 (m, 1H), 2.93-3.01 (m, 1H), 1.89-1.97 (m, 2H), 1.73-1.84 (m, 2H), 1.57-1.66 (m, 1H), 1.43-1.55 (m, 1H), 1.13 (s, 9H). ¹³C NMR (125 MHz, CHLOROFORM-d) δ ppm 141.8, 128.4, 128.2, 127.3, 65.4, 58.1, 43.8, 34.4, 25.5, 24.1, 23.2. HRMS (EI) Calculated for C₁₅H₂₄NOS [M+H]: 266.1579. Found 266.1550.

### S)-2-Methyl-propane-2-sulfinic acid [(R)-1-phenyl-butyl]-amide (9p)

Following the general procedure (GP2), the reaction of Chloro N-sulfinyl ketimine (S_{S}) 1p (1.26 g, 5.0 mmol) with LiBHEt₃ (6.0 mL, 6.0 mmol, 1.0 M in THF) affords amide 9p (1.20 g, 95%) as a viscous liquid, [α]²⁵_{D}=+ 89.37° (c 1.05, MeOH). ¹H NMR (501 MHz, CHLOROFORM-d) δ ppm 7.31-7.35 (m, 2H), 7.26-7.30 (m, 3H), 4.35-4.40 (m, 1H), 3.36 (br. s., 1H), 1.72-1.83 (m, 2H), 1.24-1.36 (m, 1H), 1.19 (s, 9H), 0.89 (t, J=7.25 Hz, 3H). ¹³C NMR (125 MHz, CHLOROFORM-d) δ ppm 142.1, 128.3, 127.6, 127.5, 59.2, 55.4, 40.9, 22.5, 19.2, 13.8. HRMS (EI) Calculated for C₁₄H₂₄NOS [M+H]: 254.1579. Found 254.1569.

### (S)-2-Methyl-propane-2-sulfinic acid [(S)-1-phenyl-butyl]-amide (10p)

Following the general procedure (GP3), the reaction of Chloro N-sulfinyl ketimine (S_{S}) 1p (1.26 g, 5.0 mmol) with DIBAL-H (6.0 mL, 6.0 mmol, 1.0 M in Toluene) and followed by LiHMDS (7.5 ml, 7.5 mmol, 1.0 mL in THF) affords amide 10p (1.18 g, 93%) as a viscous liquid, [α]²⁵_{D}=+20.19° (c 1.02, MeOH). ¹H NMR (501 MHz, CHLOROFORM-d) δ ppm 7.24-7.35 (m, 5H), 4.31-4.37 (m, 1H), 3.43 (d, J=3.47 Hz, 1H), 1.94-2.04 (m, 1H), 1.67-1.75 (m, 1H), 1.21 (s, 9H), 1.10-1.20 (m, 1H), 0.88 (t, J=7.41 Hz, 3H). ¹³C NMR (125 MHz, CHLOROFORM-d) δ ppm 142.6, 128.6, 127.7, 127.1, 58.9, 55.6, 38.8, 22.6, 18.9, 13.8. HRMS (EI) Calculated for C₁₄H₂₄NOS [M+H]: 254.1579. Found 254.1574.

### Example 5

### Deprotection to Generate Single Enantiomers of 2-Substituted Pyrrolidines

Finally, dissolving 2a in methanol followed by treatment with 4 M HCl for 30 minutes leads to cleavage of the sulfinyl group resulting in the formation of the (R)-2-(4-bromophenyl)pyrrolidine (11a) as a single enantiomer in 98% yield (Scheme 8). The other diastereomer 3a also undergoes smooth deprotection to afford (S)-2-(4-bromophenyl)pyrrolidine (ent-11a) in 97% yield. The chloro substituted pyrrolidine 2h and 3h is also tested under these conditions and gives similar results. In the same way, treatment of 2l or 3l with 4 M HCl (dioxane) in methanol for 30 minutes affords the (S)-2-(4-bromophenyl)pyrrolidine (11l) or (R)-2-(4-bromophenyl)pyrrolidine (ent-11l) in 95% and 92% yields, respectively. The absolute configuration of (S)-11l and (R)-ent-11l is confirmed by comparing the specific rotation data with literature data. (See, e.g., Kim, M-J.; Lim, G-B,; Whang, S-Y.; Ku, B-C.; Choi, J-Y. Biooranic & Medicinal Chemistry Letters, 1996, 6, 71. (b) Kostyanovsky, R. G.; Gella, I. M.; Markov, V. I.; Samojlova, Z. E. Tetrahedron 1974, 30, 39.)

Thus, the sulfinyl group can be cleaved readily under mild acidic conditions to provide the respective amines in excellent yields.

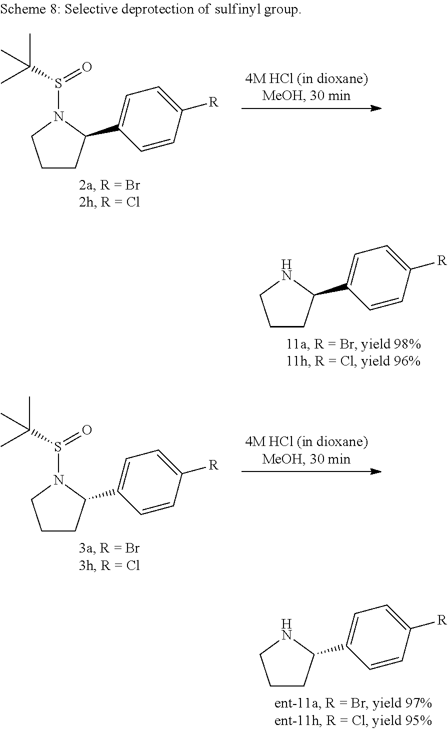

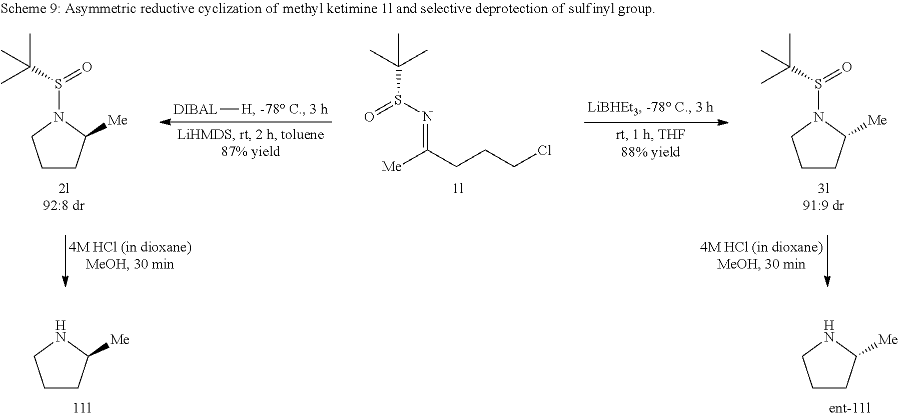

### General Procedure (GP4) for the Deprotection of Tert-Butanesulfinyl Group from 2 and 3

To a solution of 2 or 3 (2 mmol) in MeOH (10 mL) is added 4 M HCl solution (in dioxane, 2 mL). After the mixture is stirred at room temperature for 30 minutes, the mixture is concentrated to dryness and carefully dissolved in water (20 mL). The aqueous layer is washed with ethyl acetate (2×20 mL) and neutralized with 6N NaOH solution to pH ˜13. Then, the resulting aqueous solution is extracted with ethyl acetate (3×30 mL). The combined organic layers are washed with brine and then dried over anhydrous Na₂SO₄. The organic layer is concentrated to dryness to obtained pure 2-substituted pyrrolidines 11 or enti-11.

### (R)-2-(4-Bromophenyl)-pyrrolidine (11a)

Following the general procedure (GP4), the reaction of 2a (658 mg, 2.0 mmol) with 4 M HCl solution (in dioxane, 2 mL) yields the (R)-2-(4-bromophenyl)-pyrrolidine 11a (440 mg, 97%) as viscous oil, [α]²⁵_{D}=+52.1° (c 1.01, CH₂Cl₂). ¹H NMR (501 MHz, CHLOROFORM-d) δ ppm 7.40 (d, J=8.51 Hz, 2H) 7.22 (d, J=8.20 Hz, 2H) 4.05 (t, J=7.72 Hz, 1H) 3.10-3.24 (m, 1H) 2.92-3.04 (m, 1H) 2.08-2.23 (m, 1H) 1.72-2.01 (m, 3H) 1.43-1.67 (m, 1H). ¹³C NMR (125 MHz, CHLOROFORM-d) δ 144.1, 131.2, 128.2, 120.2, 61.8, 46.9, 34.4, 25.5. HRMS (EI) Calculated for C₁₀H₁₃BrN [M+H]: 226.0231. Found 226.0217.

### (S)-2-(4-Bromophenyl)-pyrrolidine (enti-11a)

Following the general procedure (GP4), the reaction of 3a (658 mg, 2.0 mmol) with 4 M HCl solution (in dioxane, 2 mL) yields the (S)-2-(4-bromophenyl)pyrrolidine enti-11a (442 mg, 98%) as viscous oil, [α]²⁵_{D}=−51.9°. (c 1.27, CH₂Cl₂). ¹H NMR (501 MHz, CHLOROFORM-d) δ ppm 7.41 (d, J=8.20 Hz, 2H) 7.22 (d, J=8.20 Hz, 2H) 4.05 (t, J=7.57 Hz, 1H) 3.11-3.21 (m, 1H) 2.91-3.05 (m, 1H) 2.08-2.27 (m, 2H) 1.69-1.98 (m, 2H) 1.43-1.67 (m, 1H). ¹³C NMR (125 MHz, CHLOROFORM-d) δ 144.0, 131.3, 126.2, 120.3, 61.8, 46.9, 34.4, 25.5. HRMS (EI) Calculated for C₁₀H₁₃BrN [M+H]: 226.0231. Found 226.0185.

### (R)-2-(4-chlorophenyl)-pyrrolidine (11h)

Following the general procedure (GP4), the reaction of 2h (570 mg, 2.0 mmol) with 4 M HCl solution (in dioxane, 2 mL) yields the (R)-2-(4-chlorophenyl)-pyrrolidine 11h (347 mg, 96%) as viscous oil, [α]²⁵_{D}=+41.1° (c 1.05, CH₂Cl₂). ¹H NMR (501 MHz, CHLOROFORM-d) δ ppm 1.49-1.71 (m, 1H) 1.70-2.03 (m, 3H) 2.08-2.27 (m, 1H) 2.90-3.07 (m, 1H) 3.09-3.27 (m, 1H) 4.09 (t, J=7.72 Hz, 1H) 7.21-7.38 (m, 4 H). ¹³C NMR (125 MHz, CHLOROFORM-d) δ ppm 143.5, 132.2, 128.9, 127.8, 61.8, 46.9, 34.4, 25.5. HRMS (EI) Calculated for C₁₀H₁₃ClN [M+H]: 182.0697. Found 182.0696.

### (S)-2-(4-chlorophenyl)-pyrrolidine (enti-11h)

Following the general procedure (GP4), the reaction of 3h (570 mg, 2.0 mmol) with 4 M HCl solution (in dioxane, 2 mL) yields the (S)-2-(4-bromophenyl)pyrrolidine enti-11h (343 mg, 95%) as viscous oil, [α]²⁵_{D}=−40.9°. (c 1.15, CH₂Cl₂). ¹H NMR (501 MHz, CHLOROFORM-d) δ ppm 1.45-1.69 (m, 1H) 1.74-1.99 (m, 3H) 2.10-2.25 (m, 1H) 2.85-3.08 (m, 1H) 3.11-3.28 (m, 1H) 4.09 (t, J=7.72 Hz, 1H) 7.21-7.36 (m, 4H). ¹³C NMR (125 MHz, CHLOROFORM-d) δ ppm 143.6, 132.2, 128.3, 127.8, 61.8, 46.9, 34.5, 25.5. HRMS (EI) Calculated for C₁₀H₁₃ClN [M+H]: 182.0702. Found 182.0737.

### (R)-2-methyl-pyrrolidine (enti-11l)

Following the general procedure (GP4), the reaction of 3l (378 mg, 2.0 mmol) with 4 M HCl solution (in dioxane, 2 mL) yields the (R)-2-methyl-pyrrolidine ent-11l (138 mg, 92%) as viscous oil, [α]²⁵_{D}=−32.1° (c 1.19, hexane), 1H NMR (501 MHz, CHLOROFORM-d) δ ppm 1.18 (m and d, 4H) 1.59 (br. s., 1H) 1.66-1.82 (m, 2H) 1.81-1.92 (m, 1H) 2.82 (q, J=8.41 Hz, 1H) 2.97-3.13 (m, 2H). ¹³C NMR (125 MHz, CHLOROFORM-d) δ ppm 54.5, 46.7, 33.7, 25.7, 21.2. HRMS (EI) Calculated for C₅H₁₂N [M+H]: 86.0954. Found 86.0970.

### (S)-2-methyl-pyrrolidine (11l)

Following the general procedure (GP4), the reaction of 2l (378 mg, 2.0 mmol) with 4 M HCl solution (in dioxane, 2 mL) yields the (R)-2-methyl-pyrrolidine 11l (142 mg, 95%) as viscous oil, [α]²⁵_{D}=+30.9°. (c 1.02, hexane), ¹H NMR (501 MHz, CHLOROFORM-d) δ ppm 2.97-3.13 (m, 2H) 2.76-2.88 (m, 1H) 1.81-1.93 (m, 1H) 1.56-1.81 (m, 3H) 1.07-1.25 (m, 4H). ¹³C NMR (125 MHz, CHLOROFORM-d) δ ppm 54.4, 46.7, 33.6, 25.6, 21.1. HRMS (EI) Calculated for C₅H₁₂N [M+H]: 86.0947. Found 86.0970.

What is claimed is:

1. A method for preparing a diastereomerically pure (S_{S}R)-2-substituted pyrrolidine of Formula (Ia) 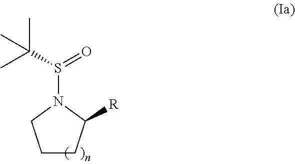
said method comprising reductive cyclization of an (S_{S})-γ-chloro-N-tert-butanesulfinyl ketimine of Formula (II) 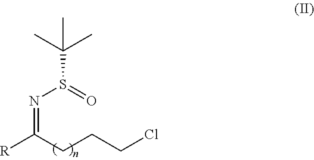
in a suitable solvent with a reducing agent selected from the group consisting of lithiumtriethylborohydride (LiBHEt₃) or L-Selectride, at a temperature of about ⁻78 to 23° C., followed by warming to room temperature and stirring for a sufficient period of time, wherein n is 0 or 1, and R is a substituted or unsubstituted alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, and sulfoxide.

2. The method of claim 1 wherein R is selected from the group consisting of: 4-BrC₆H₄, C₆H₅, 4-MeC₆H₄, 4-MeOC₆H₄, 4-tBuC₆H₄, 4-HOC₆H₄, 3-MeOC₆H₄, 4-ClC₆H₄, 4-FC₆H₄, 2-thienyl, C₆H₁₁ and Me.

3. A method for preparing a diastereomerically pure (S_{S}S)-2-substituted pyrrolidine of Formula (Ib) 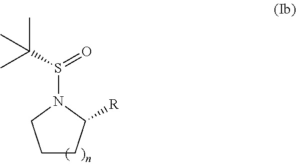
said method comprising reductive cyclization of (S_{S})-γ-chloro-N-tert-butanesulfinyl ketimines of Formula (II) 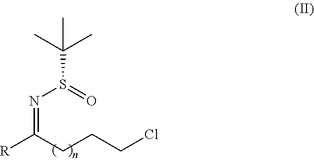
in a suitable solvent with the reducing agent diisobutylaluminum-hydride (DIBAL-H) in the presence of a strong base selected from the group consisting of lithium hexamethyldisilazide (LiHMDS), butyllithium (BuLi), sodium hydride (NaH), triethylamine (CH₂CH₃)₃N or NEt₃) and potassium hydroxide (KOH) at a temperature of about ⁻78 to 0° C. for a period of about 3 hours to 12 hours, followed by warming to room temperature and stirring for a sufficient period of time, wherein the suitable solvent is selected from the group consisting of:
(ix) toluene or tetrahydrofuran when the strong base is LiHMDS,
(x) THF when the strong base is BuLi or NaH, and
(xi) acetonitrile when the strong base is NEt₃, and
(xii) THF—H₂O when the strong base is KOH,
and wherein n is 0 or 1, and R is a substituted or unsubstituted alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, sulfoxide.

4. The method of claim 6 wherein R is selected from the group consisting of: 4-BrC₆H₄, C₆H₅, 4-MeC₆H₄, 4-MeOC₆H₄, 4-tBuC₆H₄, 4-HOC₆H₄, 3-MeOC₆H₄, 4-ClC₆H₄, 4-FC₆H₄, 2-thienyl, C₆H₁₁ and Me.

5. A method for preparing a diastereomerically pure (S_{R}S)-2-substituted pyrrolidine of Formula (Ic) 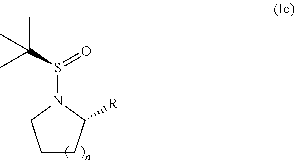
said method comprising reductive cyclization of an (S_{R})-γ-chloro-N-tert-butanesulfinyl ketimine of Formula (IIa) 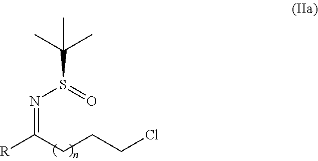
in a suitable solvent with a reducing agent selected from the group consisting of lithiumtriethylborohydride (LiBHEt₃) or L-Selectride, at a temperature of about −78 to 23° C. for a period of about 3 hours to 12 hours, followed by warming to room temperature and stirring for a sufficient period of time, and wherein n is 0 or 1, and R is a substituted or unsubstituted alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide or sulfoxide.

6. The method of claim 5 wherein R is selected from the group consisting of: 4-BrC₆H₄, C₆H₅, 4-MeC₆H₄, 4-MeOC₆H₄, 4-tBuC₆H₄, 4-HOC₆H₄, 3-MeOC₆H₄, 4-ClC₆H₄, 4-FC₆H₄, 2-thienyl, C₆H₁₁ and Me.

7. A method for preparing an diastereomerically pure (S_{R}R)-2-substituted pyrrolidine of Formula (Id) 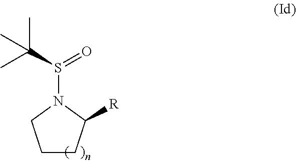
said method comprising reductive cyclization of (S_{R})-γ-chloro-N-tert-butanesulfinyl ketimines of Formula (IIa) 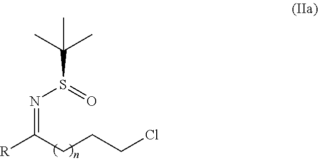
in a suitable solvent with the reducing agent diisobutylaluminum-hydride (DIBAL-H) in the presence of a strong base selected from the group consisting of lithium hexamethyldisilazide (LiHMDS), butyllithium (BuLi), sodium hydride (NaH), triethylamine (CH₂CH₃)₃N or NEt₃) and potassium hydroxide (KOH) at a temperature of about ⁻78 to 0° C. for a period of about 3 hours to 12 hours, followed by warming to room temperature and stirring for a sufficient period of time, wherein the suitable solvent is selected from the group consisting of:
(xiii) toluene or tetrahydrofuran when the strong base is LiHMDS,
(xiv) THF when the strong base is BuLi or NaH, and
(xv) acetonitrile when the strong base is NEt₃, and
(xvi) THF—H₂O when the strong base is KOH,
and wherein n is 0 or 1, and R is a substituted or unsubstituted alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, sulfoxide.

8. The method of claim 7 wherein R is selected from the group consisting of: 4-BrC₆H₄, C₆H₅, 4-MeC₆H₄, 4-MeOC₆H₄, 4-tBuC₆H₄, 4-HOC₆H₄, 3-MeOC₆H₄, 4-ClC₆H₄, 4-FC₆H₄, 2-thienyl, C₆H₁₁ and Me.

9. A method for preparing an diastereomerically pure (S_{S},R)-2-substituted piperidine of Formula (IIIa) 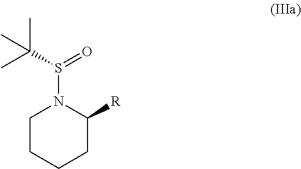
said method comprising reductive cyclization of (S_{S})-δ-chloro-N-tert-butanesulfinyl ketimine of Formula (IV) 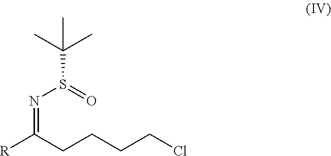
in a suitable solvent with a reducing agent selected from the group consisting of lithiumtriethylborohydride (LiBHEt₃) and L-Selectride at a temperature of about ⁻78 to 23° C., followed by warming to about room temperature and stirring for a sufficient period of time, wherein R is a substituted or unsubstituted alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, sulfoxide, sulfone, or sulfonyl.

10. The method of claim 9 wherein R is a phenyl group.

11. A method for preparing a diastereomerically pure (S_{S},S)-2-substituted piperidine of Formula (IIIb) 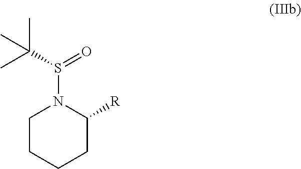
said method comprising reductive cyclization of (S_{S})-δ-chloro-N-tert-butanesulfinyl ketimine of Formula (IV) 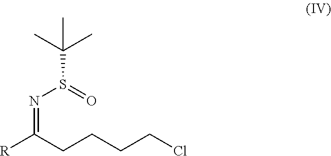
in a suitable solvent with the reducing agent DIBAL-H in the presence of a strong base and at a temperature of about ⁻78 to 0° C., followed by warming to about room temperature and stirring for a sufficient amount of time, wherein R is a substituted or unsubstituted alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, sulfoxide, sulfone, or sulfonyl.

12. The method of claim 11 wherein R is a phenyl group.

13. A method for preparing a diastereomerically pure (S_{R},S)-2-substituted piperidine of Formula (IIIc) 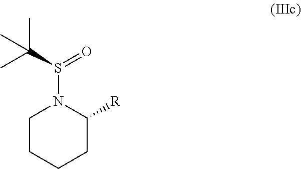
said method comprising reductive cyclization of (S_{R})-δ-chloro-N-tert-butanesulfinyl ketimine of Formula (IVa) 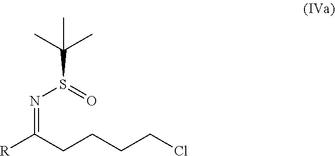
in a suitable solvent with a reducing agent selected from the group consisting of lithiumtriethylborohydride (LiBHEt₃) and L-Selectride at a temperature of about ⁻78 to 23° C., followed by warming to about room temperature and stirring for a sufficient period of time, wherein R is a substituted or unsubstituted alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, sulfoxide, sulfone, or sulfonyl.

14. The method of claim 13 wherein R is a phenyl group.

15. A method for preparing a diastereomerically pure (S_{R},R)-2-substituted piperidine of Formula (IIId) 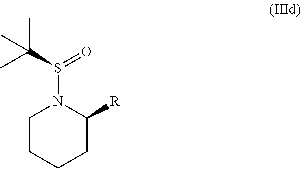
said method comprising reductive cyclization of (S_{S})-δ-chloro-N-tert-butanesulfinyl ketimine of Formula (IVa) 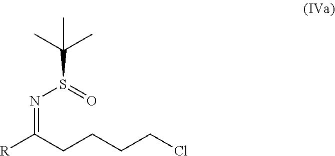
in a suitable solvent with the reducing agent DIBAL-H in the presence of a strong base and at a temperature of about ⁻78 to 0° C., followed by warming to about room temperature and stirring for a sufficient amount of time, wherein R is a substituted or unsubstituted alkyl, cycloalkyl, bridged cycloalkyl, cycloalkylalkyl, alkenyl, cycloalkenyl, cycloalkenylalkyl, alkynyl, aryl, aralkyl, arylalkyl, heterocyclyl, heteroaryl, heterocyclylalkyl, heteroaralkyl, halogen, haloalkyl, alkoxy, alkenoxy, cycloalkoxy, aryloxy, aralkyloxy, heterocyclyloxy, heterocyclylalkoxy, amide, amido, urethane, amine, amino, sulfonamido, sulfonamide, thiol, sulfide, sulfoxide, sulfone, or sulfonyl.
